(19) United States
(12) Patent Application Publication
(10) Pub. No.: US 2017/0360726 A1
(43) Pub. Date: Dec. 21, 2017

(54) COMPOUNDS, COMPOSITIONS AND METHODS OF USE

(71) Applicant: Aquinnah Pharmaceuticals, Inc., Cambridge, MA (US)

(72) Inventors: Glenn R. Larsen, Sudbury, MA (US); Manfred Weigele, Cambridge, MA (US); Joseph P. Vacca, Telford, PA (US)

(21) Appl. No.: 15/533,354

(22) PCT Filed: Dec. 4, 2015
(86) PCT No.: PCT/US2015/064107

§ 371 Date: Jun. 5, 2017

Related U.S. Application Data
(60) Provisional application No. 62/088,298, filed on Dec. 5, 2014.
(60) Provisional application No. 62/241,623, filed on Oct. 14, 2015.

(51) Int. Cl. A61K 31/18; A61K 31/4025; A61K 31/44; C07D 213/71

(52) U.S. Cl. CPC A61K 31/18; C07D 213/71; A61K 31/4025; A61K 31/44

(57) ABSTRACT

Herein, compounds, compositions and methods for modulating inclusion formation and stress granules in cells related to the onset of neurodegenerative diseases, musculoskeletal diseases, cancer, ophthalmological diseases, and viral infections are described.

### FIELD OF THE INVENTION

The invention relates to compounds, compositions and methods for modulating inclusion formation and stress granules in cells, and for treatment of neurodegenerative diseases, musculoskeletal diseases, cancer, ophthalmological diseases, and viral infections.

### BACKGROUND OF THE INVENTION

One of the hallmarks of many neurodegenerative diseases is the accumulation of protein inclusions in the brain and central nervous system. These inclusions are insoluble aggregates of proteins and other cellular components that cause damage to cells and result in impaired function. Proteins such as tau, α-synuclein, huntingtin and β-amyloid have all been found to form inclusions in the brain and are linked to the development of a number of neurodegenerative diseases, including Alzheimer's disease and Huntington's disease. Recently, the TDP-43 protein was identified as one of the major components of protein inclusions that typify the neurogenerative diseases amyotrophic lateral sclerosis (ALS) and frontotemporal lobar dementia with ubiquitin inclusions (FTLD-U) (Ash, P. E., et al. (2010) Hum Mol Genet 19(16):3206-3218; Hanson, K. A., et al. (2010) J Biol Chem 285:11068-11072; Li, Y., et al. (2010) Proc Natl Acad Sci U.S.A. 107(7):3169-3174; Neumann, M., et al. (2006) Science 314:130-133; Tsai, K. J., et al. (2010) J Exp Med 207:1661-1673; Wils, H., et al. (2010) Proc Natl Acad Sci U.S.A. 170:3858-3863). Abnormalities in TDP-43 biology appear to be sufficient to cause neurodegenerative disease, as studies have indicated that mutations in TDP-43 occur in familial ALS (Barmada, S. J., et al. (2010) J Neurosci 30:639-649; Gitcho, M. A., et al. (2008) Ann Neurol 63(4): 535-538; Johnson, B. S., et al. (2009) J Biol Chem 284:20329-20339; Ling, S. C., et al. (2010) Proc Natl Acad Sci U.S.A. 107:13318-13323; Sreedharan, J., et al. (2008) Science 319:1668-1672). In addition, TDP-43 has been found to play a role in the stress granule machinery (Colombrita, C., et al. (2009) J Neurochem 111(4):1051-1061; Liu-Yesucevitz, L., et al. (2010) PLoS One 5(10):e13250). Analysis of the biology of the major proteins that accumulate in other neurodegenerative diseases has lead to major advances in our understanding of the pathophysiology of TDP-43 inclusions as well as the development of new drug discovery platforms.

Currently, it is believed that aggregates that accumulate in neurodegenerative diseases like ALS, FTLD-U, Parkinson's disease and Huntington's disease accumulate slowly and are very difficult to disaggregate or perhaps can't be disaggregated. Thus, there is a need in the art for compostions and methods that can rapidly disaggregate stress granules and/or inhibit their formation altogether.

### SUMMARY OF THE INVENTION

In one aspect, the present invention features a compound of Formula (I):

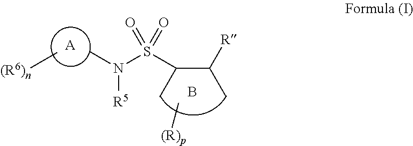

or a pharmaceutically acceptable salt thereof, wherein Ring A is aryl or heteroaryl; Ring B is 6-membered aryl or 5- or 6-membered heteroaryl; R″ is H or C(O)R¹; R¹ is C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cycloalkyl, heterocyclyl, aryl, heteroaryl, arylalkyl, heteroarylalkyl, cycloalkylalkyl, heterocyclylalkyl, —OR^{A}, —NR^{B}R^{C}, —NR^{B}C(O)R^{D}, or —SR^{E}, each of which is optionally substituted with 1-5 R⁷; each R is independently H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cyano, nitro, cycloalkyl, heterocyclyl, aryl, heteroaryl, arylalkyl, heteroarylalkyl, cycloalkylalkyl, heterocyclylalkyl, —OR^{A}, —NR^{B}R^{C}, —C(O)R^{D}, —C(O)OR^{A}, —C(O)NR^{B}R^{C}, —NR^{B}C(O)R^{D}, —NR^{B}C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁸; R⁵ is H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, —C(O)R^{D}, —C(O)OR^{A}, —C(O)NR^{B}R^{C}, —OR^{A}, or —SR^{E}, each of which is optionally substituted with 1-5 R⁹; or R⁵, together with the nitrogen atom to which it is attached, forms a heterocyclyl or heteroaryl ring with Ring A, optionally substituted with 1-3 R⁹; each R⁶ is independently C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, cycloalkylalkyl, heterocyclylalkyl, cyano, hydroxy, halo, —OR^{A}, —NR^{B}R^{C}, —C(O)R^{D}, —C(O)OR^{A}, —C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁹; each R^{A}, R^{B}, R^{C}, R^{D}, or R^{E} is independently H, C₁-C₆ alkyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, cycloalkylalkyl, heterocycloalkyl, each of which is optionally substituted with 1-4 occurrences of R⁷; or R^{B} and R^{C}, together with the atoms to which each is attached, form a heterocyclyl ring optionally substituted with 1-4 R⁷; each R⁷, R⁸, or R⁹ is independently C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, halo, cyano, nitro, —OR^{a}, —NR^{b}R^{c}, —C(O)R^{d}, —C(O)OR^{a}, —C(O)NR^{b}R^{c}, —NR^{b}C(O)R^{d}, —NR^{b}C(O)NR^{b}R^{c}, —SR^{e}, —S(O)R^{e}, —S(O)₂R^{e}, —NR^{b}S(O)₂R^{e}, or —S(O)₂NR^{b}R^{c}, each of which is optionally substituted with 1-5 R¹⁰ each R¹⁰ is C₁-C₆ alkyl, C₁-C₆ heteroalkyl, C₁-C₆ haloalkyl, C₁-C₆ hydroxyalkyl, C₁-C₆ alkoxy, C₁-C₆ haloalkoxy, cycloalkyl, heterocyclyl, aryl, heteroaryl, halo, hydroxy, cyano, or nitro, each of which is optionally substituted with 1-4 R¹; each R^{a}, R^{b}, R^{c}, R^{d}, or R^{e} is H, C₁-C₆ alkyl, cycloalkyl, heterocyclyl, aryl, or heteroaryl, each of which is optionally substituted with R¹¹; or R^{B} and R^{C}, together with the atoms to which each is attached, form a cycloalkyl or heterocyclyl ring optionally substituted with 1-4 R¹¹; each R¹¹ is independently C₁-C₆ alkyl, halo, hydroxy, cycloalkyl, alkoxy, keto, cyano, or nitro; n is 0, 1, 2, 3, 4, or 5; and p is 0, 1, or 2.

In some embodiments, Ring B is not

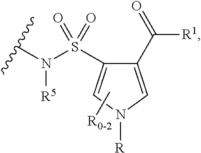

wherein the connection to C(O)R¹ and N(R⁵)S(O)₂— is as shown.

In some embodiments, R″ is H. In some embodiments, R″ is C(O)R¹.

In some embodiments, R¹ is C₁-C₆ alkyl, C₂-C₆ alkenyl, cycloalkyl, —OR^{A}, or —NR^{B}R^{C} and R^{B} and R^{C}, together with the atoms to which each is attached, form a heterocyclyl ring optionally substituted with 1-4 R⁷. In some embodiments, R¹ is —NR^{B}R^{C}, and R^{B} and R^{C} together with the atoms to which each is attached, form a heterocyclyl ring optionally substituted with 1-4 R⁷. In some embodiments, R¹ is selected from the group consisting of

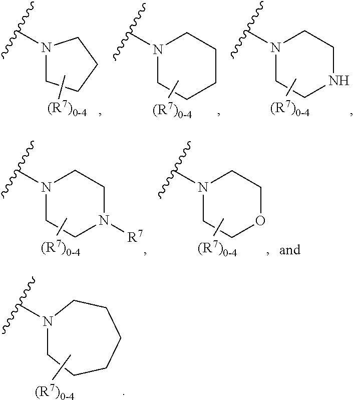

In some embodiments, R¹ is

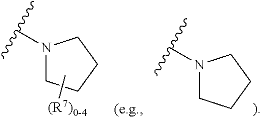

In some embodiments, each R is independently H, C₁-C₆ alkyl, C₂-C₆ alkenyl, C₁-C₆ heteroalkyl, C₁-C₆ haloalkyl, C₁-C₆ hydroxyalkyl, halo, cycloalkyl, heterocyclyl, aryl, heteroaryl, —OR^{A}, —NR^{B}R^{C}, —C(O)R^{D}, —C(O)OR^{A}, —C(O)NR^{B}R^{C}, or —NR^{B}C(O)R^{D}, each of which is optionally substituted with 1-5 R⁸. In some embodiments, each R is independently H, C₁-C₆ alkyl, C₁-C₆ haloalkyl, or halo, each of which is optionally substituted with 1-5 R⁸. In some embodiments, R is H, C₁-C₆ alkyl, C₂-C₆ alkenyl, C₁-C₆ heteroalkyl, C₁-C₆ hydroxyalkyl, cycloalkyl, heterocyclyl, aryl, or heteroaryl, each of which is optionally substituted with 1-5 R⁸. In some embodiments, R is H or C₁-C₆ alkyl, optionally substituted with 1-5 R⁸.

In some embodiments, R⁵ is H, C₁-C₆ alkyl, C₂-C₆ alkenyl, C₁-C₆ heteroalkyl, C₁-C₆ haloalkyl, C₁-C₆ hydroxyalkyl, cycloalkyl, heterocyclyl, aryl, or heteroaryl, each of which is optionally substituted with 1-5 R⁹. In some embodiments, R⁵ is H or C₁-C₆ alkyl (e.g., H).

In some embodiments, Ring A is a monocyclic or bicyclic aryl or heteroaryl. In some embodiments, Ring A is monocyclic or bicyclic aryl. In some embodiments, Ring A is phenyl. In some embodiments, Ring A is a 5- or 6-membered heteroaryl. In some embodiments, Ring A is selected from the group consisting of pyrrolyl, pyrazolyl, imidazolyl, triazinyl, furanyl, oxazolyl, isoxazolyl, thiophenyl, thiazolyl, isothiazolyl, oxadiazolyl, thiadiazolyl, dithiazolyl, pyridyl, pyrimidinyl, pyrazinyl, pyridazinyl, triazinyl, and tetrazinyl.

In some embodiments, R⁶ is C₁-C₆ alkyl, C₂-C₆ alkenyl, C₁-C₆ heteroalkyl, C₁-C₆ haloalkyl, C₁-C₆ hydroxyalkyl, cycloalkyl, heterocyclyl, aryl, heteroaryl, cyano, hydroxy, halo, —OR^{A}, —NR^{B}R^{C}, —C(O)R^{D}, —C(O)OR^{A}, —C(O)NR^{B}R^{C}, —S(O)₂R^{E}, —NR^{B}S(O)₂R^{E}, or —S(O)₂NR^{B}R^{C}, each of which is optionally substituted with 1-5 R⁹. In some embodiments, R⁶ is C₁-C₆ alkyl, cyano, hydroxy, halo, —OR^{A}, or —NR^{B}R^{C}. In some embodiments, R⁶ is C₁-C₆ alkyl.

In some embodiments, Ring A is selected from the group consisting of:

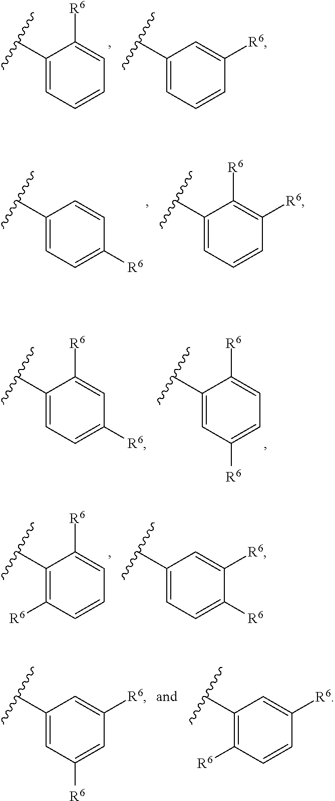

In some embodiments, Ring A is selected from

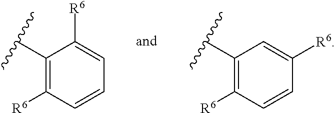

In some embodiments, Ring A is

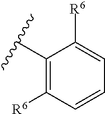

and R⁶ is C₁-C₆ alkyl or halo.
In some embodiments, Ring B is a 6-membered aryl. In some embodiments, Ring B is phenyl.
In some embodiments, Ring B is selected from

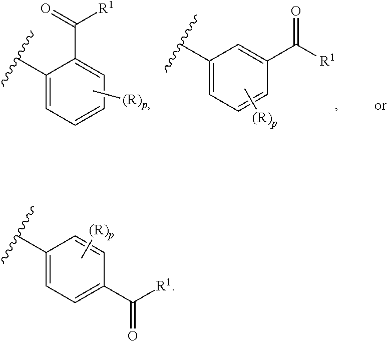

In some embodiments, Ring B is a 5- or 6-membered heteroaryl. In some embodiments, Ring B is selected from the group consisting of pyrrolyl, pyrazolyl, imidazolyl, triazinyl, furanyl, oxazolyl, isoxazolyl, thiophenyl, thiazolyl, isothiazolyl, oxadiazolyl, thiadiazolyl, dithiazolyl, pyridyl, pyrimidinyl, pyrazinyl, pyridazinyl, triazinyl, and tetrazinyl. In some embodiments, Ring B is selected from

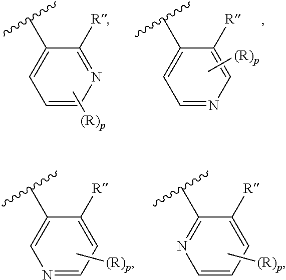

wherein the connectivity to —S(O)₂ is indicated by a wavy line and the connectivity to R″ is as shown.

In some embodiments, n is 0, 1, or 2 (e.g., 2).

In some embodiments, p is 0 or 1 (e.g., 0).

In some embodiments, the compound of Formula (I) is a compound of Formula (Ib):

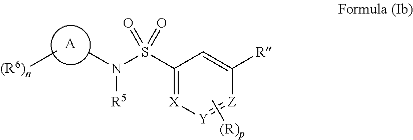

or a pharmaceutically acceptable salt thereof, wherein each of X, Y, and Z is independently N or C(R^{D})₂, and Ring A, R, R″, R⁵, R⁶, R^{D}, n, p and subvariables thereof are defined as for Formula (I).

In some embodiments, the compound of Formula (I) is a compound of Formula (Ic):

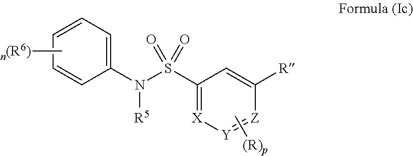

or a pharmaceutically acceptable salt thereof, wherein each of X, Y, and Z is independently N or C(R^{D} 2, and R, R″, R⁵, R⁶, R^{D}, n, p and subvariables thereof are defined as for Formula (I).

In some embodiments, the compound of Formula (I) is a compound of Formula (Id):

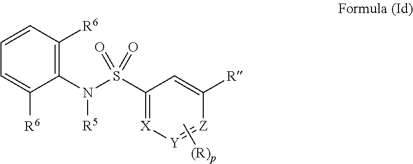

or a pharmaceutically acceptable salt thereof, wherein each of X, Y, and Z is independently N or C(R^{D})₂, and R, R¹, R⁵, R⁶, R^{D}, n, p and subvariables thereof are defined as for Formula (I).

In some embodiments, the compound of Formula (I) is a compound of Formula (Ie):

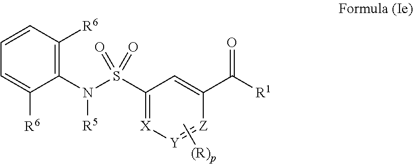

or a pharmaceutically acceptable salt thereof, wherein each of X, Y, and Z is independently N or C(R^{D})₂, and R, R¹, R⁵, R⁶, R^{D}, p and subvariables thereof are defined as for Formula (I).

In some embodiments, the compound of Formula (I) is selected from the group consisting of:

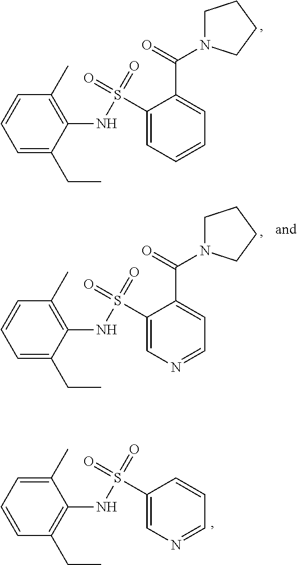 [SMILES: CCc1cccc(C)c1NS(=O)(=O)c1ccccc1C(=O)N1CCCC1 ; CCc1cccc(C)c1NS(=O)(=O)c1cnccc1C(=O)N1CCCC1 ; CCc1cccc(C)c1NS(=O)(=O)c1cccnc1]

or a pharmaceutically acceptable salt thereof.

In another aspect, the present invention features a pharmaceutical composition comprising at least one compound according to Formula (I) (e.g., Formula (Ia), Formula (Ib), Formula (Ic), Formula (Id), or Formula (Ie)), or a pharmaceutically acceptable salt thereof, in a mixture with a pharmaceutically acceptable excipient, diluent or carrier.

In another aspect, the present invention features a composition for use in modulating stress granules comprising contacting a cell with a compound of Formula (I). In some embodiments, stress granule formation is inhibited. In some embodiments, stress granule is disaggregated. In some embodiments, stress granule formation is stimulated. In some embodiments, stress granule comprises tar DNA binding protein-43 (TDP-43), T-cell intracellular antigen 1 (TIA-1), TIA1 cytotoxic granule-associated RNA binding protein-like 1 (TIAR), GTPase activating protein binding protein 1 (G3BP-1), GTPase activating protein binding protein 2 (G3BP-2), tris tetraprolin (TTP), fused in sarcoma (FUS), or fragile X mental retardation protein (FMRP).

In another aspect, the present invention features a composition for use in modulating TDP-43 inclusion formation comprising contacting a cell with a compound of Formula (I). In some embodiments, TDP-43 inclusion formation is inhibited. In some embodiments, the TDP-43 inclusion is disaggregated. In some embodiments, TDP-43 inclusion formation is stimulated.

In some embodiments, the composition is administered to a subject suffering from a neurodegenerative disease or disorder, a musculoskeletal disease or disorder, a cancer, an ophthalmological disease or disorder, and/or a viral infection, the method comprising administering a compound of Formula (I) to a subject in need thereof.

In some embodiments, wherein the neurodegenerative disease is selected from the group consisting of Alzheimer's disease, frontotemporal dementia (FTD), FTLD-U, FTD caused by mutations in the progranulin protein or tau protein (e.g., progranulin-deficient FTLD), frontotemporal dementia with inclusion body myopathy (IBMPFD), frontotemporal dementia with motor neuron disease, amyotrophic lateral sclerosis (ALS), Huntington's disease (HD), Huntington's chorea, prion diseases (e.g., Creutzfeld-Jacob disease, bovine spongiform encephalopathy, Kuru, or scrapie), Lewy Body disease, diffuse Lewy body disease (DLBD), polyglutamine (polyQ)-repeat diseases, trinucleotide repeat diseases, cerebral degenerative diseases, presenile dementia, senile dementia, Parkinsonism linked to chromosome 17 (FTDP-17), progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, HIV-associated dementia, Parkinson's disease, Parkinson's disease with dementia, dementia with Lewy bodies, Down's syndrome, multiple system atrophy, spinal muscular atrophy (SMA, e.g., SMA Type I (e.g., Werdnig-Hoffmann disease) SMA Type II, SMA Type III (e.g., Kugelberg-Welander disease), or congenital SMA with arthrogryposis), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, age-related disorders and dementias, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), autism, or any combination thereof.

In some embodiments, the musculoskeletal disease is selected from the group consisting of muscular dystrophy, facioscapulohumeral muscular dystrophy (e.g., FSHD1 or FSHD2), Freidrich's ataxia, progressive muscular atrophy (PMA), mitochondrial encephalomyopathy (MELAS), multiple sclerosis, inclusion body myopathy, inclusion body myositis (e.g., sporadic inclusion body myositis), post-polio muscular atrophy (PPMA), motor neuron disease, myotonia, myotonic dystrophy, sacropenia, multifocal motor neuropathy, inflammatory myopathies, and paralysis.

In some embodiments, the cancer is selected from the group consisting of breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, ophthalmological cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, vulval cancer, or any combination thereof. In some embodiments, the non-Hodgkin's lymphoma is selected from a B-cell lymphoma or a T-cell lymphoma. In some embodiments, the B-cell or T-cell lymphoma is selected from the group consisting of diffuse large B-cell lymphoma, primary mediastinal B-cell lymphoma, intravascular large B-cell lymphoma, follicular lymphoma, chronic lymphocytic leukemia/small lymphocytic lymphoma, mantle cell lymphoma, marginal zone B-cell lymphomas, extranodal marginal B-cell lymphomas, mucosa-associated lymphoid tissue (MALT) lymphomas, modal marginal zone B-cell lymphoma, splenic marginal zone B-cell lymphoma, Burkitt lymphoma, lymphoplasmacytic lymphoma, Waldenström's macroglobulinemia, hairy cell leukemia, primary central nervous system (CNS) lymphoma, precursor T-lymphoblastic lymphomalleukemia, peripheral T-cell lymphoma, cutaneous T-cell lymphoma, smoldering adult T-cell lymphoma, chronic adult T-cell lymphoma, acute adult T-cell lymphoma, lymphomatous adult T-cell lymphoma, angioimmunoblastic T-cell lymphoma, extranodal natural killer T-cell lymphoma nasal type (ENKL), enteropathy-associated intestinal T-cell lymphoma (EATL), and anaplastic large cell lymphoma (ALCL).

In some embodiments, the ophthalmological disease is selected from the group consisting of macular degeneration, age-related macular degeneration, diabetes retinopathy, histoplasmosis, macular hole, macular pucker, Bietti's crystalline dystrophy, retinal detachment, retinal thinning, retinoblastoma, retinopathy of prematurity, Usher's syndrome, vitreous detachment, Refsum disease, retinitis pigmentosa, onchocerciasis, choroideremia, Leber congenital amaurosis, retinoschisis, juvenile retinoschisis, Stargardt disease, ophthalmoplegia, or any combination thereof.

In some embodiments, the viral infection is caused by a virus selected from the group consisting of West Nile virus, respiratory syncytial virus (RSV), herpes simplex virus 1, herpes simplex virus 2, Epstein-Barr virus (EBV), hepatitis virus A, hepatitis virus B, hepatitis virus C, influenza viruses, chicken pox, avian flu viruses, smallpox, polio viruses, HIV-1, HIV-2, Ebola virus, and any combination thereof.

In some embodiments, the subject is a mammal. In some embodiments, the subject is human.

In some embodiments, use of the composition further comprises the step of diagnosing the subject with the neurodegenerative disease or disorder, musculoskeletal disease or disorder, cancer, ophthalmological disease or disorder, or viral infection prior to onset of said administration.

In some embodiments, pathology of said neurodegenerative disease or disorder, said musculoskeletal disease or disorder, said cancer, said ophthalmological disease or disorder, and said viral infection comprises stress granules. In some embodiments, pathology of said neurodegenerative disease, said musculoskeletal disease or disorder, said cancer, said ophthalmological disease or disorder, and said viral infection comprises TDP-43 inclusions.

In another aspect, the invention provides methods for treatment of a neurodegenerative disease or disorder, a musculoskeletal disease or disorder, a cancer, an ophthalmological disease or disorder (e.g., a retinal disease or disorder), and/or a viral infection in a subject, the method comprising administering a compound of Formula (I) to a subject in need thereof.

In another aspect, the invention provides methods of diagnosing a neurodegenerative disease in a subject, the method comprising administering a compound of Formula (I) to the subject. For use in diagnosis, the compound of Formula (I) can be modified with a label.

In another aspect, the invention provides methods of modulating stress granules comprising contacting a cell with a compound of Formula (I).

In another aspect, the invention provides methods of modulating TDP-43 inclusion formation comprising contacting a cell with a compound of Formula (I).

In another aspect, the invention provides a method of screening for modulators of TDP-43 aggregation comprising contacting a compound of Formula (I) with the cell that expresses TDP-43 and develops spontaneous inclusions.

Still other objects and advantages of the invention will become apparent to those of skill in the art from the disclosure herein, which is simply illustrative and not restrictive. Thus, other embodiments will be recognized by the skilled artisan without departing from the spirit and scope of the invention.

### DETAILED DESCRIPTION OF THE INVENTION

Amyotrophic lateral sclerosis (ALS), also known as Lou Gehrig's disease or Charcot disease, is a fatal neurodegenerative disease that occurs with an incidence of approximately 1/100,000 (Mitchell, J. D. and Borasio, G. D., (2007) Lancet 369:2031-41). There is currently no therapy for ALS, and the average survival rate of patients from the onset of the disease is roughly four years. ALS presents with motor weakness in the distal limbs that rapidly progresses proximally (Mitchell, J. D. and Borasio, G. D., (2007) Lancet 369:2031-41; Lambrechts, D. E., et al. (2004) Trends Mol Med 10:275-282). Studies over the past decade have indicated that TDP-43 is the major protein that accumulates in affected motor neurons in sporadic ALS (Neumann, M., et al. (2006) Science 314:130-133). The causes of sporadic ALS are not known, but identification of the major pathological species accumulating in the spinal cord of ALS patients represents a seminal advance for ALS research. To date, TDP-43 is the only protein that has been both genetically and pathologically linked with sporadic ALS, which represents the predominant form of the disease. Multiple papers have identified mutations in TDP-43 associated with sporadic and familial ALS (Sreedharan, J., et al. (2008) Science 319:1668-1672; Gitcho, M. A., et al. (2008) Ann Neurol 63(4):535-538; Neumann, M., et al. (2006) Science 314:130-133). Inhibitors of cell death and inclusions linked to TDP-43 represent a novel therapeutic approach to ALS, and may also elucidate the biochemical pathway linked to the formation of TDP-43 inclusions (Boyd, J. B., et al. (2014) J Biomol Screen 19(1):44-56). As such, TDP-43 represents one of the most promising targets for pharmacotherapy of ALS.

TDP-43 is a nuclear RNA binding protein that translocates to the cytoplasm in times of cellular stress, where it forms cytoplasmic inclusions. These inclusions then colocalize with reversible protein-mRNA aggregates termed “stress granules” (SGs) (Anderson P. and Kedersha, N. (2008) Trends Biochem Sci 33:141-150; Kedersha, N. and Anderson, P. (2002) Biochem Soc Trans 30:963-969; Lagier-Tourenne, C., et al. (2010) Hum Mol Genet 19:R46-R64). Under many stress-inducing conditions (e.g., arsenite treatment, nutrient deprivation), TDP-43 co-localization with SGs approaches 100%. The reversible nature of SG-based aggregation offers a biological pathway that can be applied to reverse the pathology and toxicity associated with TDP-43 inclusion formation. Studies show that agents that inhibit SG formation also inhibit formation of TDP-43 inclusions (Liu-Yesucevitz, L., et al. (2010) PLoS One 5(10):e13250). The relationship between TDP-43 and stress granules is important because it provides a novel approach for dispersing TDP-43 inclusions using physiological pathways that normally regulate this reversible process, rather than direct physical disruption of protein aggregation by a small molecule pharmaceutical. Investigating the particular elements of the SG pathway that regulate TDP-43 inclusion formation can identify selective approaches for therapeutic intervention to delay or halt the progression of disease. Stress granule biology also regulates autophagy and apoptosis, both of which are linked to neurodegeneration. Hence, compounds inhibiting TDP-43 aggregation may play a role in inhibiting neurodegeneration.

### Modulators of TDP-43 Inclusions and Stress Granules

Accordingly, in one aspect, the invention provides a compound of Formula (I):

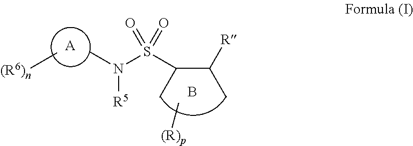

or a pharmaceutically acceptable salt thereof, wherein:

Ring A is aryl or heteroaryl;

Ring B is 6-membered aryl or 5- or 6-membered heteroaryl;

R″ is H or C(O)R¹;

R¹ is C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cycloalkyl, heterocyclyl, aryl, heteroaryl, arylalkyl, heteroarylalkyl, cycloalkylalkyl, heterocyclylalkyl, —OR^{A}, —NR^{B}R^{C}, —NR^{B}C(O)R^{D}, or —SR^{E}, each of which is optionally substituted with 1-5 R⁷;

each R is independently H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cyano, nitro, cycloalkyl, heterocyclyl, aryl, heteroaryl, arylalkyl, heteroarylalkyl, cycloalkylalkyl, heterocyclylalkyl, —OR^{A}, —NR^{B}R^{C}, —C(O)R^{D}, —C(O)OR^{A}, —C(O)NR^{B}R^{C}, —NR^{B}C(O)R^{D}, —NR^{B}C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁸;

R⁵ is H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, —C(O)R^{D}, —C(O)OR^{A}, —C(O)NR^{B}R^{C}, —OR^{A}, or —SR^{E}, each of which is optionally substituted with 1-5 R⁹;

or R⁵, together with the nitrogen atom to which it is attached, forms a heterocyclyl or heteroaryl ring with Ring A, optionally substituted with 1-3 R⁹;

each R⁶ is independently C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, cycloalkylalkyl, heterocyclylalkyl, cyano, hydroxy, halo, —OR^{A}, —NR^{B}R^{C}, —C(O)R^{D}, —C(O)OR^{A}, —C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁹;

each R^{A}, R^{B}, R^{C}, R^{D}, or R^{E} is independently H, C₁-C₆ alkyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, cycloalkylalkyl, heterocycloalkyl, each of which is optionally substituted with 1-4 occurrences of R⁷; or R^{B} and R^{C}, together with the atoms to which each is attached, form a heterocyclyl ring optionally substituted with 1-4 R⁷;

each R⁷, R⁸, or R⁹ is independently C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, halo, cyano, nitro, —OR^{a}, —NR^{b}R^{c}, —C(O)R^{d}, —C(O)OR^{a}, —C(O)NR^{b}R^{c}, —NR^{b}C(O)R^{d}, —NR^{b}C(O)NR^{b}R^{c}, —SR^{e}, —S(O)R^{e}, —S(O)₂R^{e}, —NR^{b}S(O)₂R^{e}, or —S(O)₂NR^{b}R^{c}, each of which is optionally substituted with 1-5 R¹⁰;

each R¹⁰ is C₁-C₆ alkyl, C₁-C₆ heteroalkyl, C₁-C₆ haloalkyl, C₁-C₆ hydroxyalkyl, C₁-C₆ alkoxy, C₁-C₆ haloalkoxy, cycloalkyl, heterocyclyl, aryl, heteroaryl, halo, hydroxy, cyano, or nitro, each of which is optionally substituted with 1-4 R¹¹;

each R^{a}, R^{b}, R^{c}, R^{d}, or R^{e} is H, C₁-C₆ alkyl, cycloalkyl, heterocyclyl, aryl, or heteroaryl, each of which is optionally substituted with R^{U}; or R^{B} and R^{C}, together with the atoms to which each is attached, form a cycloalkyl or heterocyclyl ring optionally substituted with 1-4 R¹¹;

each R¹¹ is independently C₁-C₆ alkyl, halo, hydroxy, cycloalkyl, alkoxy, keto, cyano, or nitro;

n is 0, 1, 2, 3, 4, or 5; and

p is 0, 1, or 2.

In some embodiments, Ring B is not

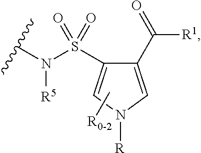

wherein the connection to C(O)R¹ and N(R⁵)S(O)₂— is as shown.

In some embodiments, R″ is H. In some embodiments, R″ is C(O)R¹.

In some embodiments, R¹ is C₁-C₆ alkyl, C₂-C₆ alkenyl, cycloalkyl, —OR^{A}, or —NR^{B}R^{C} and R^{B} and R^{C}, together with the atoms to which each is attached, form a heterocyclyl ring optionally substituted with 1-4 R⁷. In some embodiments, R¹ is —NR^{B}R^{C}, and R^{B} and R^{C}, together with the atoms to which each is attached, form a heterocyclyl ring optionally substituted with 1-4 R⁷. In some embodiments, R¹ is selected from the group consisting of

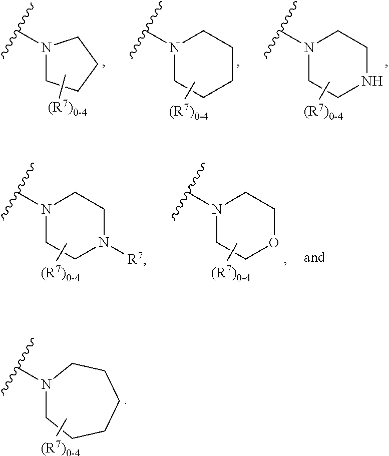

In some embodiments, R¹ is

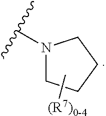

In some embodiments, R¹ is

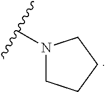 [SMILES: *N1CCCC1]

In some embodiments, R″ is C(O)R¹ and R¹ is —NR^{B}R^{C}, and R^{B} and R^{C}, together with the atoms to which each is attached, form a heterocyclyl ring optionally substituted with 1-4 R⁷. In some embodiments, R″ is C(O)R¹ and R¹ is selected from the group consisting of

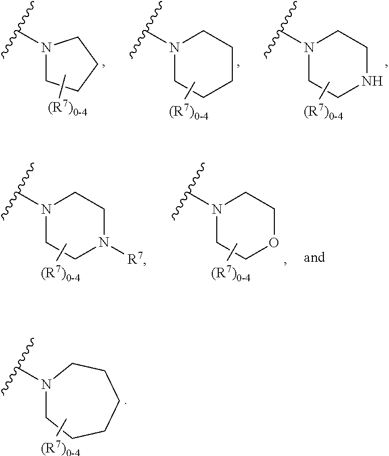

In some embodiments, R″ is C(O)R¹ and R¹ is

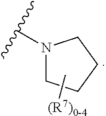

In some embodiments, each R is independently H, C₁-C₆ alkyl, C₂-C₆ alkenyl, C₁-C₆ heteroalkyl, C₁-C₆ haloalkyl, C₁-C₆ hydroxyalkyl, halo, cycloalkyl, heterocyclyl, aryl, heteroaryl, —OR^{A}, —NR^{B}R^{C}, —C(O)R^{D}, —C(O)OR^{A}, —C(O)NR^{B}R^{C}, or —NR^{B}C(O)R^{D}, each of which is optionally substituted with 1-5 R⁸. In some embodiments, each R is independently H, C₁-C₆ alkyl, C₁-C₆ haloalkyl, or halo, each of which is optionally substituted with 1-5 R⁸. In some embodiments, R is H, C₁-C₆ alkyl, C₂-C₆ alkenyl, C₁-C₆ heteroalkyl, C₁-C₆ hydroxyalkyl, cycloalkyl, heterocyclyl, aryl, or heteroaryl, each of which is optionally substituted with 1-5 R⁸. In some embodiments, R is H or C₁-C₆ alkyl, optionally substituted with 1-5 R⁸.

In some embodiments, R⁵ is H, C₁-C₆ alkyl, C₂-C₆ alkenyl, C₁-C₆ heteroalkyl, C₁-C₆ haloalkyl, C₁-C₆ hydroxyalkyl, cycloalkyl, heterocyclyl, aryl, or heteroaryl, each of which is optionally substituted with 1-5 R⁹. In some embodiments, R⁵ is H or C₁-C₆ alkyl. In some embodiments, R⁵ is H.

In some embodiments, Ring A is aryl. In some embodiments, Ring A is monocyclic or bicyclic aryl. In some embodiments, Ring A is monocyclic aryl. In some embodiments, Ring A is bicyclic aryl. In some embodiments, Ring A is a 6-membered aryl. In some embodiments, Ring A is phenyl.

In some embodiments, R⁶ is C₁-C₆ alkyl, C₂-C₆ alkenyl, C₁-C₆ heteroalkyl, C₁-C₆ haloalkyl, C₁-C₆ hydroxyalkyl, cycloalkyl, heterocyclyl, aryl, heteroaryl, cyano, hydroxy, halo, —OR^{A}, —NR^{B}R^{C}, —C(O)R^{D}, —C(O)OR^{A}, —C(O)NR^{B}R^{C}, —S(O)₂R^{E}, —NR^{B}S(O)₂R^{E}, or —S(O)₂NR^{B}R^{C}, each of which is optionally substituted with 1-5 R⁹. In some embodiments, R⁶ is C₁-C₆ alkyl, cyano, hydroxy, halo, —OR^{A}, or —NR^{B}R^{C}. In some embodiments, R⁶ is C₁-C₆ alkyl or halo. In some embodiments, R⁶ is C₁-C₆ alkyl. In some embodiments, R⁶ is methyl, ethyl, or isopropyl.

In some embodiments, Ring A is selected from the group consisting of:

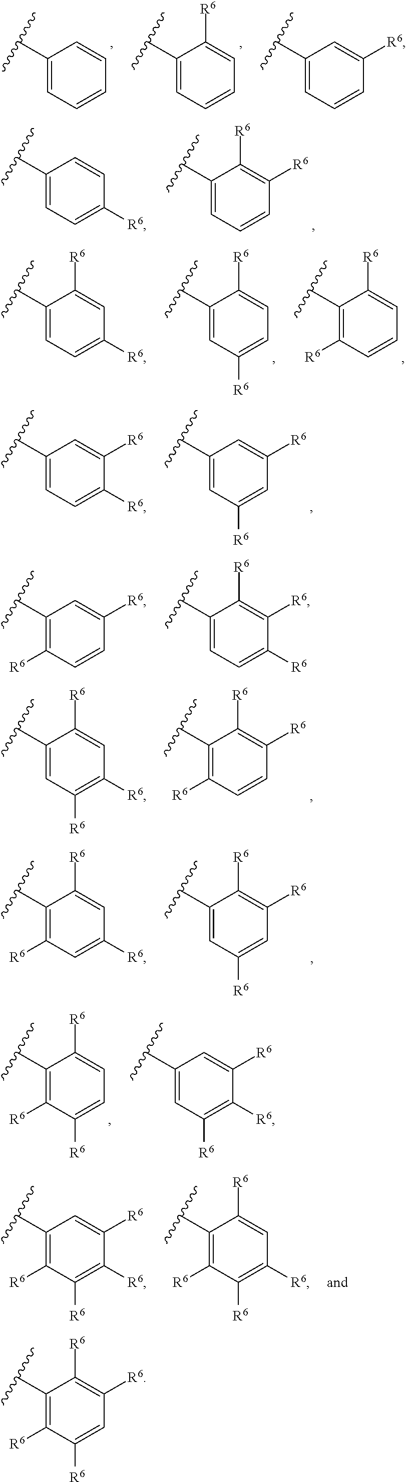

In some embodiments, Ring A is selected from the group consisting of:

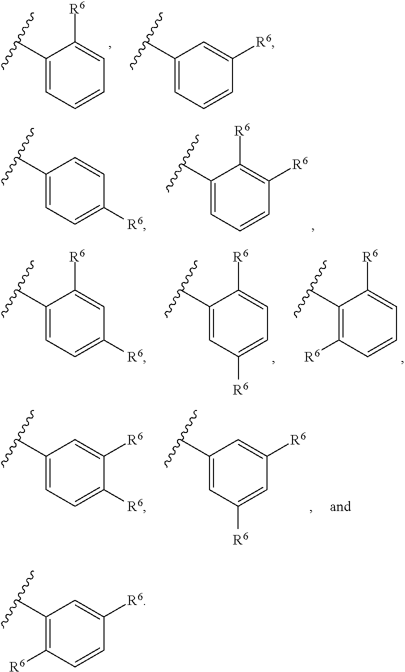

In some embodiments, Ring A is selected from the group consisting of:

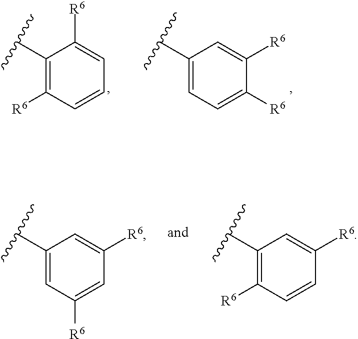

In some embodiments, Ring A is selected from

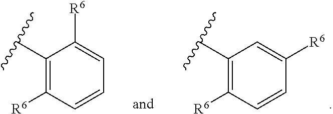

In some embodiments, Ring A is

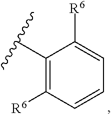

and R⁶ is C₁-C₆ alkyl or halo.

In some embodiments, Ring A is 2-ethyl-6-methylphenyl. In some embodiments, Ring A is

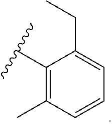 [SMILES: *c1c(C)cccc1CC]

In some embodiments, Ring A is a monocyclic or bicyclic heteroaryl. In some embodiments, Ring A is a 5- or 6-membered monocyclic heteroaryl. In some embodiments, Ring A is selected from the group consisting of pyrrolyl, pyrazolyl, imidazolyl, triazinyl, furanyl, oxazolyl, isoxazolyl, thiophenyl, thiazolyl, isothiazolyl, oxadiazolyl, thiadiazolyl, dithiazolyl, pyridyl, pyrimidinyl, pyrazinyl, pyridazinyl, triazinyl, and tetrazinyl.

In some embodiments, Ring B is a 6-membered aryl. In some embodiments, Ring B is phenyl.

In some embodiments, Ring B is selected from

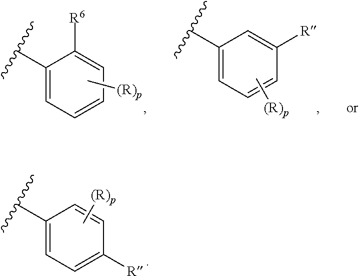

In some embodiments, Ring B is selected from

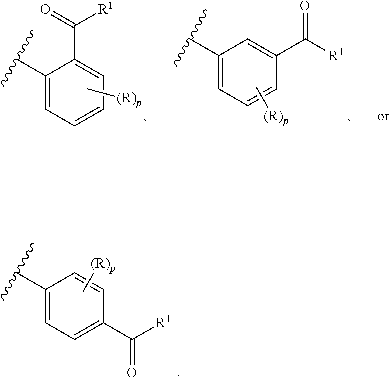

In some embodiments, Ring B is a 5- or 6-membered heteroaryl. In some embodiments, Ring B is selected from the group consisting of pyrrolyl, pyrazolyl, imidazolyl, triazinyl, furanyl, oxazolyl, isoxazolyl, thiophenyl, thiazolyl, isothiazolyl, oxadiazolyl, thiadiazolyl, dithiazolyl, pyridyl, pyrimidinyl, pyrazinyl, pyridazinyl, triazinyl, and tetrazinyl. In some embodiments, Ring B is selected from the group consisting of pyridyl, pyrimidinyl, pyrazinyl, and pyridazinyl. In some embodiments, Ring B is pyridyl.

In some embodiments, Ring B is selected from

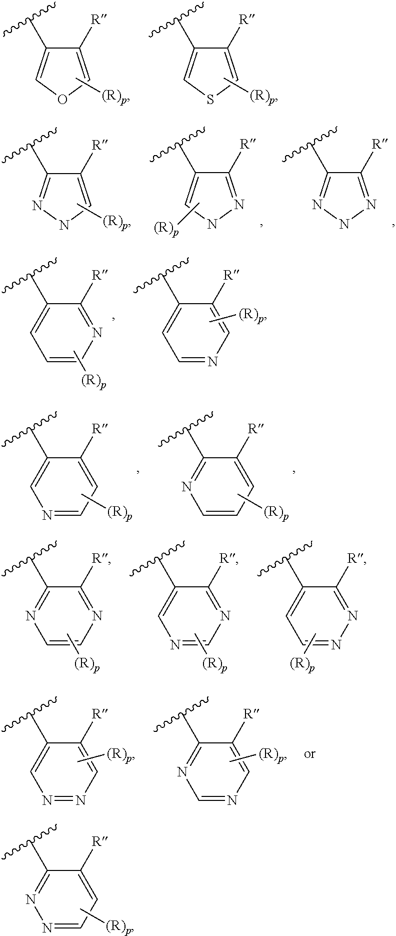

wherein the connectivity to —S(O)₂ is indicated by a wavy line and the connectivity to R″ is as shown. In some embodiments, Ring B is selected from

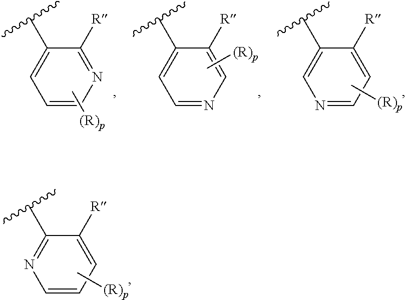

wherein the connectivity to —S(O)₂ is indicated by a wavy line and the connectivity to R″ is as shown. In some embodiments, Ring B is

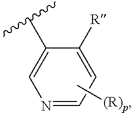

wherein the connectivity to —S(O)₂ is indicated by a wavy line and the connectivity to R″ is as shown.

In some embodiments, n is 0, 1, or 2. In some embodiments, n is 0 or 1. In some embodiments, n is 0. In some embodiments, n is 1. In some embodiments, n is 2.

In some embodiments, p is 0 or 1. In some embodiments, p is 0. In some embodiments, p is 1.

In some embodiments, the compound of Formula (I) is a compound of Formula (Ia):

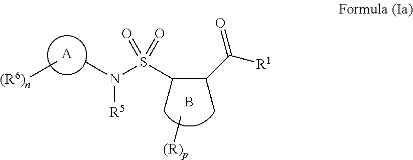

or a pharmaceutically acceptable salt thereof, wherein:

Ring A is aryl or heteroaryl;

Ring B is 6-membered aryl or 5- or 6-membered heteroaryl;

R¹ is C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cycloalkyl, heterocyclyl, aryl, heteroaryl, arylalkyl, heteroarylalkyl, cycloalkylalkyl, heterocyclylalkyl, —OR^{A}, —NR^{B}R^{C}, —NR^{B}C(O)R^{D}, or —SR^{E}, each of which is optionally substituted with 1-5 R⁷;

each R is independently H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cyano, nitro, cycloalkyl, heterocyclyl, aryl, heteroaryl, arylalkyl, heteroarylalkyl, cycloalkylalkyl, heterocyclylalkyl, —OR^{A}, —NR^{B}R^{C}, —C(O)R^{D}, —C(O)OR^{A}, —C(O)NR^{B}R^{C}, —NR^{B}C(O)R^{D}, —NR^{B}C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁸;

R⁵ is H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, —C(O)R^{D}, —C(O)OR^{A}, —C(O)NR^{B}R^{C}, —OR^{A}, or —SR^{E}, each of which is optionally substituted with 1-5 R⁹;

or R⁵, together with the nitrogen atom to which it is attached, forms a heterocyclyl or heteroaryl ring with Ring A, optionally substituted with 1-3 R⁹;

each R⁶ is independently C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, cycloalkylalkyl, heterocyclylalkyl, cyano, hydroxy, halo, —OR^{A}, —NR^{B}R^{C}, —C(O)R^{D}, —C(O)OR^{A}, —C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁹;

each R^{A}, R^{B}, R^{C}, R^{D}, or R^{E} is independently H, C₁-C₆ alkyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, cycloalkylalkyl, heterocycloalkyl, each of which is optionally substituted with 1-4 occurrences of R⁷; or R^{B} and R^{C}, together with the atoms to which each is attached, form a heterocyclyl ring optionally substituted with 1-4 R⁷;

each R⁷, R⁸, or R⁹ is independently C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, halo, cyano, nitro, —OR^{a}, —NR^{b}R^{c}, —C(O)R^{d}, —C(O)OR^{a}, —C(O)NR^{b}R^{c}, —NR^{b}C(O)R^{d}, —NR^{b}C(O)NR^{b}R^{c}, —SR^{e}, —S(O)R^{e}, —S(O)₂R^{e}, —NR^{b}S(O)₂R^{e}, or —S(O)₂NR^{b}R^{c}, each of which is optionally substituted with 1-5 R¹⁰;

each R¹⁰ is C₁-C₆ alkyl, C₁-C₆ heteroalkyl, C₁-C₆ haloalkyl, C₁-C₆ hydroxyalkyl, C₁-C₆ alkoxy, C₁-C₆ haloalkoxy, cycloalkyl, heterocyclyl, aryl, heteroaryl, halo, hydroxy, cyano, or nitro, each of which is optionally substituted with 1-4 R¹¹;

each R^{a}, R^{b}, R^{c}, R^{d}, or R^{e} is H, C₁-C₆ alkyl, cycloalkyl, heterocyclyl, aryl, or heteroaryl, each of which is optionally substituted with R¹; or R^{B} and R^{C}, together with the atoms to which each is attached, form a cycloalkyl or heterocyclyl ring optionally substituted with 1-4 R¹¹;

each R¹¹ is independently C₁-C₆ alkyl, halo, hydroxy, cycloalkyl, alkoxy, keto, cyano, or nitro;

n is 0, 1, 2, 3, 4, or 5; and

p is 0, 1, or 2;

provided that Ring B is not

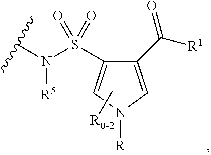

wherein the connection to C(O)R¹ and N(R⁵)S(O)₂— is as shown.

In some embodiments, R¹ is C₁-C₆ alkyl, C₂-C₆ alkenyl, cycloalkyl, —OR^{A}, or —NR^{B}R^{C} and R^{B} and R^{C}, together with the atoms to which each is attached, form a heterocyclyl ring optionally substituted with 1-4 R⁷. In some embodiments, R¹ is —NR^{B}R^{C}, and R^{B} and R^{C}, together with the atoms to which each is attached, form a heterocyclyl ring optionally substituted with 1-4 R⁷. In some embodiments, R¹ is selected from the group consisting of

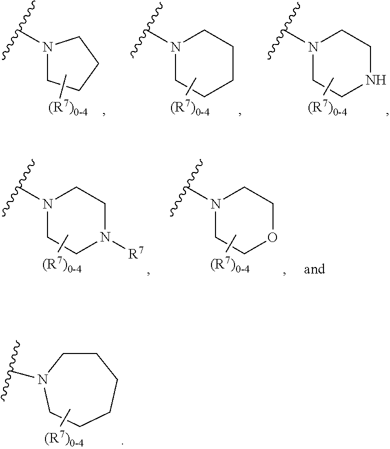

In some embodiments, R¹ is

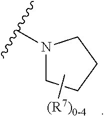

In some embodiments, R¹ is

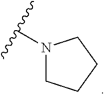 [SMILES: *N1CCCC1]

In some embodiments, each R is independently H, C₁-C₆ alkyl, C₂-C₆ alkenyl, C₁-C₆ heteroalkyl, C₁-C₆ haloalkyl, C₁-C₆ hydroxyalkyl, halo, cycloalkyl, heterocyclyl, aryl, heteroaryl, —OR^{A}, —NR^{B}R^{C}, —C(O)R^{D}, —C(O)OR^{A}, —C(O)NR^{B}R^{C}, or —NR^{B}C(O)R^{D}, each of which is optionally substituted with 1-5 R⁸. In some embodiments, each R is independently H, C₁-C₆ alkyl, C₁-C₆ haloalkyl, or halo, each of which is optionally substituted with 1-5 R⁸. In some embodiments, R is H, C₁-C₆ alkyl, C₂-C₆ alkenyl, C₁-C₆ heteroalkyl, C₁-C₆ hydroxyalkyl, cycloalkyl, heterocyclyl, aryl, or heteroaryl, each of which is optionally substituted with 1-5 R⁸. In some embodiments, R is H or C₁-C₆ alkyl, optionally substituted with 1-5 R⁸.

In some embodiments, R⁵ is H, C₁-C₆ alkyl, C₂-C₆ alkenyl, C₁-C₆ heteroalkyl, C₁-C₆ haloalkyl, C₁-C₆ hydroxyalkyl, cycloalkyl, heterocyclyl, aryl, or heteroaryl, each of which is optionally substituted with 1-5 R⁹. In some embodiments, R⁵ is H or C₁-C₆ alkyl. In some embodiments, R⁵ is H.

In some embodiments, Ring A is aryl. In some embodiments, Ring A is monocyclic or bicyclic aryl. In some embodiments, Ring A is monocyclic aryl. In some embodiments, Ring A is bicyclic aryl. In some embodiments, Ring A is a 6-membered aryl. In some embodiments, Ring A is phenyl.

In some embodiments, R⁶ is C₁-C₆ alkyl, C₂-C₆ alkenyl, C₁-C₆ heteroalkyl, C₁-C₆ haloalkyl, C₁-C₆ hydroxyalkyl, cycloalkyl, heterocyclyl, aryl, heteroaryl, cyano, hydroxy, halo, —OR^{A}, —NR^{B}R^{C}, —C(O)R^{D}, —C(O)OR^{A}, —C(O)NR^{B}R^{C}, —S(O)₂R^{E}, —NR^{B}S(O)₂R^{E}, or —S(O)₂NR^{B}R^{C}, each of which is optionally substituted with 1-5 R⁹. In some embodiments, R⁶ is C₁-C₆ alkyl, cyano, hydroxy, halo, —OR^{A}, or —NR^{B}R^{C}. In some embodiments, R⁶ is C₁-C₆ alkyl or halo. In some embodiments, R⁶ is C₁-C₆ alkyl. In some embodiments, R⁶ is methyl, ethyl, or isopropyl.

In some embodiments, Ring A is selected from the group consisting of:

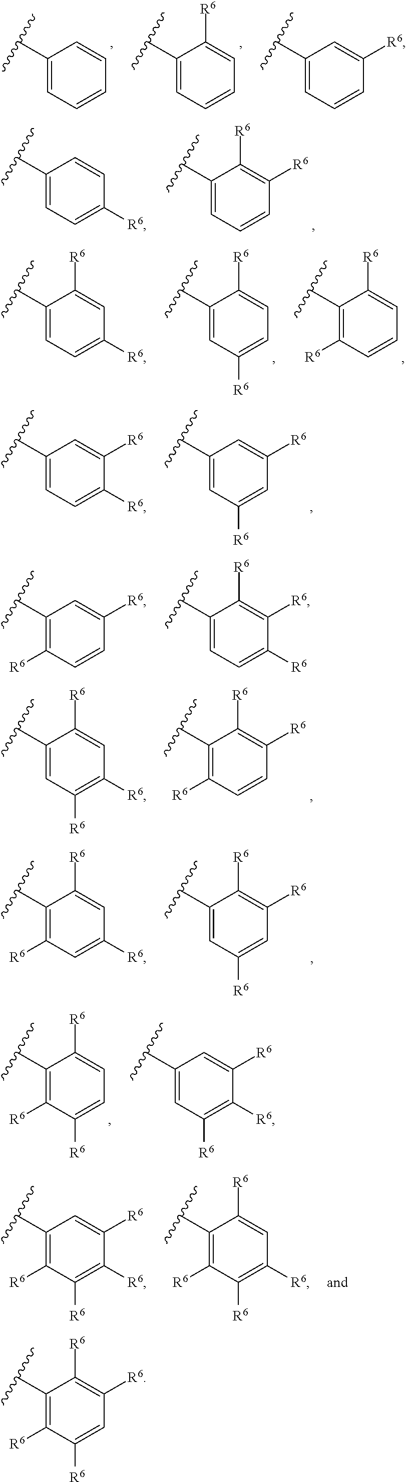

In some embodiments, Ring A is selected from the group consisting of:

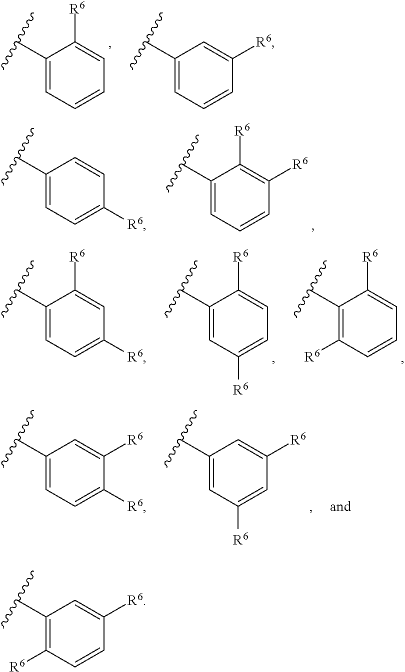

In some embodiments, Ring A is selected from the group consisting of:

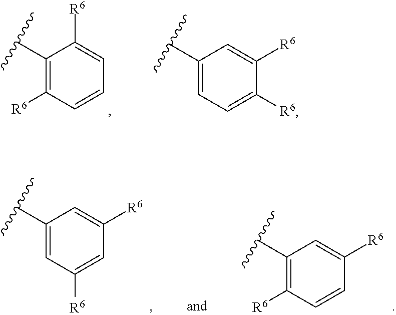

In some embodiments, Ring A is selected from

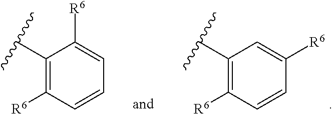

In some embodiments, Ring A is

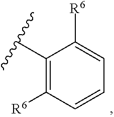

and R⁶ is C₁-C₆ alkyl or halo.

In some embodiments, Ring A is 2-ethyl-6-methylphenyl. In some embodiments, Ring A is

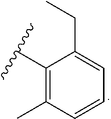 [SMILES: *c1c(C)cccc1CC]

In some embodiments, Ring A is a monocyclic or bicyclic heteroaryl. In some embodiments, Ring A is a 5- or 6-membered monocyclic heteroaryl. In some embodiments, Ring A is selected from the group consisting of pyrrolyl, pyrazolyl, imidazolyl, triazinyl, furanyl, oxazolyl, isoxazolyl, thiophenyl, thiazolyl, isothiazolyl, oxadiazolyl, thiadiazolyl, dithiazolyl, pyridyl, pyrimidinyl, pyrazinyl, pyridazinyl, triazinyl, and tetrazinyl.

In some embodiments, Ring B is a 6-membered aryl. In some embodiments, Ring B is phenyl.

In some embodiments, Ring B is a 6-membered aryl. In some embodiments, Ring B is phenyl. In some embodiments, Ring B is selected from

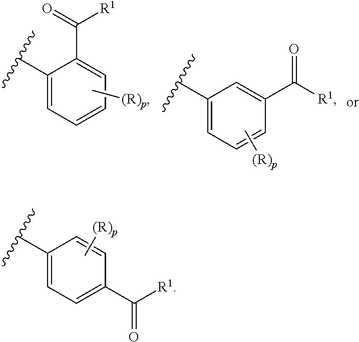

In some embodiments, Ring B is a 5- or 6-membered heteroaryl. In some embodiments, Ring A is selected from the group consisting of pyrrolyl, pyrazolyl, imidazolyl, triazinyl, furanyl, oxazolyl, isoxazolyl, thiophenyl, thiazolyl, isothiazolyl, oxadiazolyl, thiadiazolyl, dithiazolyl, pyridyl, pyrimidinyl, pyrazinyl, pyridazinyl, triazinyl, and tetrazinyl. In some embodiments, Ring B is selected from the group consisting of pyridyl, pyrimidinyl, pyrazinyl, and pyridazinyl. In some embodiments, Ring B is pyridyl.

In some embodiments, Ring B is selected from

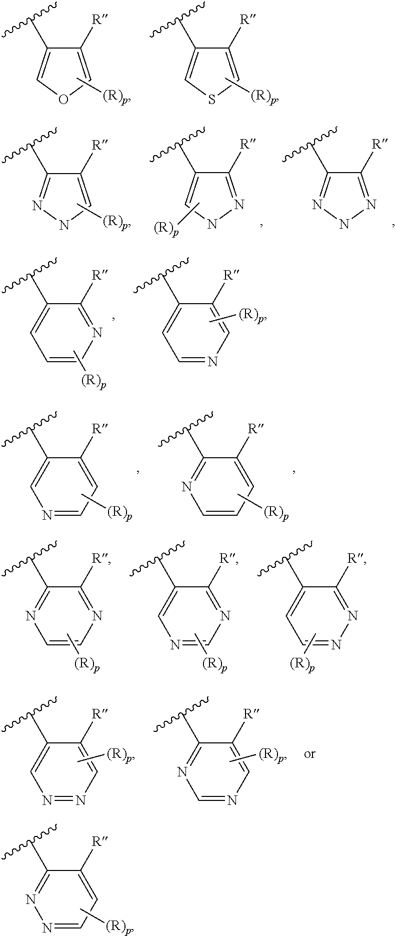

wherein the connectivity to —S(O)₂ is indicated by a wavy line and the connectivity to R″ is as shown. In some embodiments, Ring B is selected from

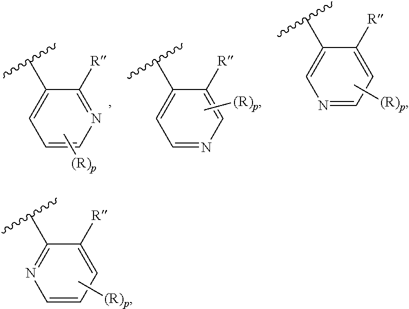

wherein the connectivity to —S(O)₂ is indicated by a wavy line and the connectivity to R″ is as shown. In some embodiments, Ring B is

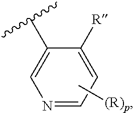

wherein the connectivity to —S(O)₂ is indicated by a wavy line and the connectivity to R″ is as shown.

In some embodiments, Ring B is

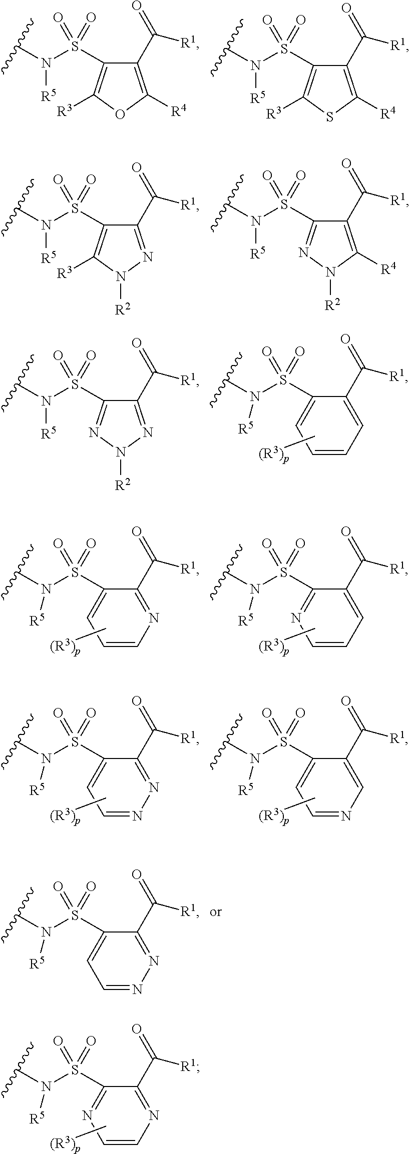

wherein the connection to C(O)R¹ and N(R⁵)S(O)₂— is as shown.

wherein R² is H, substituted C₁ alkyl, C₂-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, cycloalkylalkyl, heterocyclylalkyl, —C(O)R^{D}, —C(O)OR^{A}, —C(O)NR^{B}R^{C}, —OR^{A}, or —SR^{E}, each of which is optionally substituted with 1-5 R⁸; and each of R³ and R⁴ is independently H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cyano, nitro, cycloalkyl, heterocyclyl, aryl, heteroaryl, arylalkyl, heteroarylalkyl, cycloalkylalkyl, heterocyclylalkyl, —OR^{A}, —NR^{B}R^{C}, —C(O)R^{D}, —C(O)OR^{A}, —C(O)NR^{B}R^{C}, —NR^{B}C(O)R^{D}, —NR^{B}C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁹.

In some embodiments, R² is H, C₁-C₆ alkyl, C₂-C₆ alkenyl, C₁-C₆ heteroalkyl, C₁-C₆ hydroxyalkyl, cycloalkyl, heterocyclyl, aryl, or heteroaryl, each of which is optionally substituted with 1-5 R⁸. In some embodiments, R² is H or C₁-C₆ alkyl.

In some embodiments, each of R³ and R⁴ is independently H, C₁-C₆ alkyl, C₂-C₆ alkenyl, C₁-C₆ heteroalkyl, C₁-C₆ haloalkyl, C₁-C₆ hydroxyalkyl, halo, cycloalkyl, heterocyclyl, aryl, heteroaryl, —OR^{A}, —NR^{B}R^{C}, —C(O)R^{D}, —C(O)OR^{A}, —C(O)NR^{B}R^{C}, or —NR^{B}C(O)R^{D}, each of which is optionally substituted with 1-5 R⁹. In some embodiments, each of R³ and R⁴ is independently R³ and R⁴ is independently H, C₁-C₆ alkyl, C₁-C₆ haloalkyl, or halo.

In some embodiments, n is 0, 1, or 2. In some embodiments, n is 0 or 1. In some embodiments, n is 0. In some embodiments, n is 1. In some embodiments, n is 2.

In some embodiments, p is 0 or 1. In some embodiments, p is 0. In some embodiments, p is 1.

In some embodiments, the compound of Formula (I) is a compound of Formula (Ib):

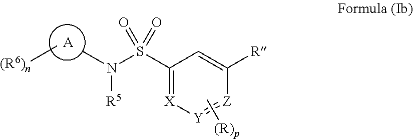

or a pharmaceutically acceptable salt thereof, wherein each of X, Y, and Z is independently N or C(R^{D})₂, and Ring A, R, R″, R⁵, R⁶, R^{D}, n, p and subvariables thereof are defined as for Formula (I).

In some embodiments, the compound of Formula (I) is a compound of Formula (Ic):

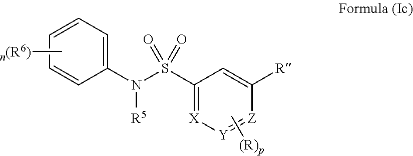

or a pharmaceutically acceptable salt thereof, wherein each of X, Y, and Z is independently N or C(R^{D})₂, and R, R″, R⁵, R⁶, R^{D}, n, p and subvariables thereof are defined as for Formula (I).

In some embodiments, the compound of Formula (I) is a compound of Formula (Id):

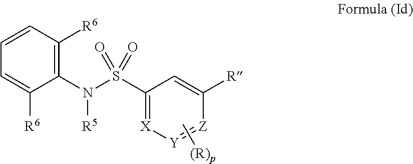

or a pharmaceutically acceptable salt thereof, wherein each of X, Y, and Z is independently N or C(R^{D})₂, and R, R¹, R⁵, R⁶, R^{D}, n, p and subvariables thereof are defined as for Formula (I).

In some embodiments, the compound of Formula (I) is a compound of Formula (Ie):

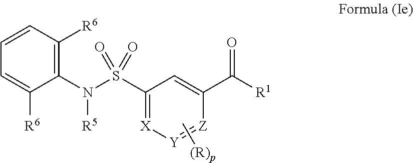

or a pharmaceutically acceptable salt thereof, wherein each of X, Y, and Z is independently N or C(R^{D})₂, and R, R¹, R⁵, R⁶, R^{D}, p and subvariables thereof are defined as for Formula (I).

In some embodiments, the compound of Formula (I) is selected from the group consisting of:

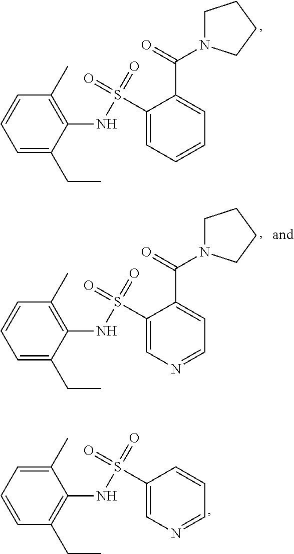 [SMILES: CCc1cccc(C)c1NS(=O)(=O)c1ccccc1C(=O)N1CCCC1 ; CCc1cccc(C)c1NS(=O)(=O)c1cnccc1C(=O)N1CCCC1 ; CCc1cccc(C)c1NS(=O)(=O)c1cccnc1]

or a pharmaceutically acceptable salt thereof.

In another aspect, the invention provides a pharmaceutical composition comprising a compound of Formula (I) or a pharmaceutically acceptable salt thereof in a mixture with a pharmaceutically acceptable excipient, diluent or carrier.

In another aspect, the invention provides a method of modulating stress granule formation, the method comprising contacting a cell with a compound of Formula (I). In some embodiments, stress granule formation is inhibited. In some embodiments, the stress granule is disaggregated. In some embodiments, stress granule formation is stimulated.

In some embodiments, a compound of Formula (I) inhibits the formation of a stress granule. The compound of Formula (I) can inhibit the formation of a stress granule by at least 10%, at least 20%, at least 30%, at least 40%, at least 50%, at least 60%, at least 70%, at least 80%, at least 90%, at least 95%, or 100% (i.e., complete inhibition) relative to a control.

In some embodiments, a compound of Formula (I) disaggregates a stress granule. The compound of Formula (I) can disperses or disaggregate a stress granule by at least 10%, at least 20%, at least 30%, at least 40%, at least 50%, at least 60%, at least 70%, at least 80%, at least 90%, at least 95%, or 100% (i.e., complete dispersal) relative to a control.

In some embodiments, the stress granule comprises tar DNA binding protein-43 (TDP-43), T-cell intracellular antigen 1 (TIA-1), TIA1 cytotoxic granule-associated RNA binding protein-like 1 (TIAR, TIAL1), GTPase activating protein binding protein 1 (G3BP-1), GTPase activating protein binding protein 2 (G3BP-2), tris tetraprolin (TTP, ZFP36), fused in sarcoma (FUS), or fragile X mental retardation protein (FMRP, FMR1).

In some embodiments, the stress granule comprises tar DNA binding protein-43 (TDP-43), T-cell intracellular antigen 1 (TIA-1), TIA1 cytotoxic granule-associated RNA binding protein-like 1 (TIAR, TIAL1), GTPase activating protein binding protein 1 (G3BP-1), GTPase activating protein binding protein 2 (G3BP-2), fused in sarcoma (FUS), or fragile X mental retardation protein (FMRP, FMR1).

In some embodiments, the stress granule comprises tar DNA binding protein-43 (TDP-43), T-cell intracellular antigen 1 (TIA-1), TIA1 cytotoxic granule-associated RNA binding protein-like 1 (TIAR, TIAL1), GTPase activating protein binding protein 1 (G3BP-1), GTPase activating protein binding protein 2 (G3BP-2), or fused in sarcoma (FUS).

In some embodiments, the stress granule comprises tar DNA binding protein-43 (TDP-43).

In some embodiments, the stress granule comprises T-cell intracellular antigen 1 (TIA-1).

In some embodiments, the stress granule comprises TIA-1 cytotoxic granule-associated RNA binding protein-like 1 (TIAR, TIAL1).

In some embodiments, the stress granule comprises GTPase activating protein binding protein 1 (G3BP-1).

In some embodiments, the stress granule comprises GTPase activating protein binding protein 2 (G3BP-2).

In some embodiments, the stress granule comprises tris tetraprolin (TTP, ZFP36).

In some embodiments, the stress granule comprises fused in sarcoma (FUS).

In some embodiments, the stress granule comprises fragile X mental retardation protein (FMRP, FMR1).

In another aspect, the invention provides a method of modulating TDP-43 inclusion formation, the method comprising contacting a cell with a compound of Formula (I). In some embodiments, TDP-43 inclusion formation is inhibited. In some embodiments, the TDP-43 inclusion is disaggregated. In some embodiments, TDP-43 inclusion formation is stimulated.

In some embodiments, a compound of Formula (I) inhibits the formation of a TDP-43 inclusion. The compound of Formula (I) can inhibit the formation of a TDP-43 inclusion by at least 10%, at least 20%, at least 30%, at least 40%, at least 50%, at least 60%, at least 70%, at least 80%, at least 90%, at least 95%, or 100% (i.e., complete inhibition) relative to a control.

In some embodiments, a compound of Formula (I) disaggregates a TDP-43 inclusion. The compound of Formula (I) can disperses or disaggregate a TDP-43 inclusion by at least 10%, at least 20%, at least 30%, at least 40%, at least 50%, at least 60%, at least 70%, at least 80%, at least 90%, at least 95%, or 100% (i.e., complete dispersal) relative to a control.

In another aspect, the invention provides a method for treatment of a neurodegenerative disease or disorder, a musculoskeletal disease or disorder, a cancer, an ophthalmological disease or disorder (e.g., a retinal disease or disorder), and/or a viral infection, the method comprising administering an effective amount of a compound of Formula (I) to a subject in need thereof.

In some embodiments, the methods are performed in a subject suffering from a neurodegenerative disease or disorder, a musculoskeletal disease or disorder, a cancer, an ophthalmological disease or disorder (e.g., a retinal disease or disorder), and/or a viral infection.

In some embodiments, the methods are performed in a subject suffering from a neurodegenerative disease or disorder. In some embodiments, the methods are performed in a subject suffering from a musculoskeletal disease or disorder. In some embodiments, the methods are performed in a subject suffering from a cancer. In some embodiments, the methods are performed in a subject suffering from an ophthalmological disease or disorder (e.g., a retinal disease or disorder). In some embodiments, the methods are performed in a subject suffering from a viral infection or viral infections.

In some embodiments, the methods comprise administering a compound of Formula (I) to a subject in need thereof. In some embodiments, the subject is a mammal. In some embodiments, the subject is a nematode. In some embodiments, the subject is human.

In some embodiments, the methods further comprise the step of diagnosing the subject with a neurodegenerative disease or disorder, a musculoskeletal disease or disorder, a cancer, an ophthalmological disease or disorder (e.g., a retinal disease or disorder), or a viral infection prior to administration of a compound of Formula (I). In some embodiments, the methods further comprise the step of diagnosing the subject with a neurodegenerative disease or disorder prior to administration of a compound of Formula (I).

In some embodiments, the neurodegenerative disease is selected from the group consisting of Alzheimer's disease, frontotemporal dementia (FTD), FTLD-U, FTD caused by mutations in the progranulin protein or tau protein (e.g., progranulin-deficient FTLD), frontotemporal dementia with inclusion body myopathy (IBMPFD), frontotemporal dementia with motor neuron disease, amyotrophic lateral sclerosis (ALS), Huntington's disease (HD), Huntington's chorea, prion diseases (e.g., Creutzfeld-Jacob disease, bovine spongiform encephalopathy, Kuru, and scrapie), Lewy Body disease, diffuse Lewy body disease (DLBD), polyglutamine (polyQ)-repeat diseases, trinucleotide repeat diseases, cerebral degenerative diseases, presenile dementia, senile dementia, Parkinsonism linked to chromosome 17 (FTDP-17), progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, HIV-associated dementia, Parkinson's disease, Parkinson's disease with dementia, dementia with Lewy bodies, Down's syndrome, multiple system atrophy, spinal muscular atrophy (SMA, e.g., SMA Type I (e.g., Werdnig-Hoffmann disease), SMA Type II, SMA Type III (e.g., Kugelberg-Welander disease), and congenital SMA with arthrogryposis), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, age-related disorders and dementias, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury) autism, other diseases or disorders relating to the aberrant expression of TDP-43 and altered proteostasis, and any combination thereof.

In some embodiments, the neurodegenerative disease is selected from the group consisting of Alzheimer's disease, frontotemporal dementia (FTD), FTLD-U, FTD caused by mutations in the progranulin protein or tau protein (e.g., progranulin-deficient FTLD), amyotrophic lateral sclerosis (ALS), Huntington's disease (HD), Huntington's chorea, Creutzfeld-Jacob disease, senile dementia, Parkinsonism linked to chromosome 17 (FTDP-17), progressive supranuclear palsy (PSP), Pick's disease, primary progressive aphasia, corticobasal dementia, Parkinson's disease, Parkinson's disease with dementia, dementia with Lewy bodies, Down's syndrome, multiple system atrophy, spinal muscular atrophy (SMA), spinocerebellar ataxia, spinal degenerative disease/motor neuron degenerative diseases, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), hippocampal sclerosis, corticobasal degeneration, Alexander disease, Cockayne syndrome, and any combination thereof.

In some embodiments, the neurodegenerative disease is frontotemporal dementia (FTD). In some embodiments, the neurodegenerative disease is Alzheimer's disease or amyotrophic lateral sclerosis (ALS).

In some embodiments, the musculoskeletal disease is selected from the group consisting of muscular dystrophy, facioscapulohumeral muscular dystrophy (e.g., FSHD1 or FSHD2), Freidrich's ataxia, progressive muscular atrophy (PMA), mitochondrial encephalomyopathy (MELAS), multiple sclerosis, inclusion body myopathy, inclusion body myositis (e.g., sporadic inclusion body myositis), post-polio muscular atrophy (PPMA), motor neuron disease, myotonia, myotonic dystrophy, sacropenia, multifocal motor neuropathy, inflammatory myopathies, paralysis, and other diseases or disorders relating to the aberrant expression of TDP-43 and altered proteostasis.

In some embodiments, compounds of Formula (I) may be used to prevent or treat symptoms caused by or relating to said musculoskeletal diseases, e.g., kyphosis, hypotonia, foot drop, motor dysfunctions, muscle weakness, muscle atrophy, neuron loss, muscle cramps, altered or aberrant gait, dystonias, astrocytosis (e.g., astrocytosis in the spinal cords), liver disease, respiratory disease or respiratory failure, inflammation, headache, and pain (e.g., back pain, neck pain, leg pain, or inflammatory pain).

In some embodiments, the cancer is selected from the group consisting of breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, ophthalmological cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, vulval cancer, and any combination thereof.

In some embodiments, the cancer is selected from the group consisting of blastoma, carcinoma, a glioblastoma, hepatic carcinoma, lymphoma, leukemia, and any combination thereof.

In some embodiments, the cancer is selected from Hodgkin's lymphoma or non-Hodgkin's lymphoma. In some embodiments, the cancer is a non-Hodgkin's lymphoma, selected from the group consisting of a B-cell lymphoma (e.g., diffuse large B-cell lymphoma, primary mediastinal B-cell lymphoma, intravascular large B-cell lymphoma, follicular lymphoma, chronic lymphocytic leukemia/small lymphocytic lymphoma, mantle cell lymphoma, marginal zone B-cell lymphomas, extranodal marginal B-cell lymphomas, mucosa-associated lymphoid tissue (MALT) lymphomas, modal marginal zone B-cell lymphoma, splenic marginal zone B-cell lymphoma, Burkitt lymphoma, lymphoplasmacytic lymphoma, Waldenström's macroglobulinemia, hairy cell leukemia, and primary central nervous system (CNS) lymphoma) and a T-cell lymphoma (e.g., precursor T-lymphoblastic lymphomalleukemia, peripheral T-cell lymphoma, cutaneous T-cell lymphoma, adult T-cell lymphoma (e.g., smoldering adult T-cell lymphoma, chronic adult T-cell lymphoma, acute adult T-cell lymphoma, lymphomatous adult T-cell lymphoma), angioimmunoblastic T-cell lymphoma, extranodal natural killer T-cell lymphoma nasal type (ENKL), enteropathy-associated intestinal T-cell lymphoma (EATL) (e.g., Type I EATL and Type II EATL), and anaplastic large cell lymphoma (ALCL)).

In some embodiments, the ophthalmological disease or disorder (e.g., retinal disease or disorder) is selected from macular degeneration (e.g., age-related macular degeneration), diabetes retinopathy, histoplasmosis, macular hole, macular pucker, Bietti's crystalline dystrophy, retinal detachment, retinal thinning, retinoblastoma, retinopathy of prematurity, Usher's syndrome, vitreous detachment, Refsum disease, retinitis pigmentosa, onchocerciasis, choroideremia, Leber congenital amaurosis, retinoschisis (e.g., juvenile retinoschisis), Stargardt disease, ophthalmoplegia, and the like.

In some embodiments, the ophthalmological disease or disorder (e.g., retinal disease or disorder) is selected from macular degeneration (e.g., age-related macular degeneration), diabetes retinopathy, histoplasmosis, macular hole, macular pucker, Bietti's crystalline dystrophy, retinoblastoma, retinopathy of prematurity, Usher's syndrome, Refsum disease, retinitis pigmentosa, onchocerciasis, choroideremia, Leber congenital amaurosis, retinoschisis (e.g., juvenile retinoschisis), Stargardt disease, and the like.

In some embodiments, the viral infection is caused by a virus selected from the group consisting of West Nile virus, respiratory syncytial virus (RSV), herpes simplex virus 1, herpes simplex virus 2, Epstein-Barr virus (EBV), hepatitis virus A, hepatitis virus B, hepatitis virus C, influenza viruses, chicken pox, avian flu viruses, smallpox, polio viruses, HIV-1, HIV-2, Ebola virus, and any combination thereof.

In some embodiments, the viral infection is caused by a virus selected from the group consisting of herpes simplex virus 1, herpes simplex virus 2, Epstein-Barr virus (EBV), hepatitis virus A, hepatitis virus B, hepatitis virus C, HIV-1, HIV-2, Ebola virus, and any combination thereof.

In some embodiments, the viral infection is HIV-1 or HIV-2.

In some embodiments, the pathology of the neurodegenerative disease or disorder, musculoskeletal disease or disorder, cancer, ophthalmological disease or disorder (e.g., retinal disease or disorder), and/or viral infection comprises stress granules.

In some embodiments, pathology of the disease or disorder comprises stress granules. By comprising stress granules is meant that number of stress granules in a cell in the subject is changed relative to a control and/or healthy subject or relative to before onset of said disease or disorder. Exemplary diseases and disorders pathology of which incorporate stress granules include, but are not limited to, neurodegenerative diseases, musculoskeletal diseases, cancers, ophthalmological diseases (e.g., retinal diseases), and viral infections.

In another aspect, the invention provides methods of diagnosing a neurodegenerative disease, a musculoskeletal disease, a cancer, an ophthalmological disease (e.g., a retinal disease), or a viral infection in a subject, the method comprising administering a compound of Formula (I) to the subject. In some embodiments, the invention provides methods of diagnosing a neurodegenerative disease in a subject, the method comprising administering a compound of Formula (I) to the subject. For use in diagnosis, a compound of Formula (I) can be modified with a label.

In another aspect, the invention provides methods of modulating stress granules comprising contacting a cell with a compound of Formula (I).

In another aspect, the invention provides methods of modulating TDP-43 inclusion formation comprising contacting a cell with a compound of Formula (I). In some embodiments, TDP-43 is inducibly expressed. In some embodiments, the cell line is a neuronal cell line.

In some embodiments, the cell is treated with a physiochemical stressor. In some embodiments, the physicochemical stressor is selected from arsenite, nutrient deprivation, heat shock, osmotic shock, a virus, genotoxic stress, radiation, oxidative stress, oxidative stress, a mitochondrial inhibitor, and an endoplasmic reticular stressor. In some embodiments, the physicochemical stressor is ultraviolet or x-ray radiation. In some embodiments, the physicochemical stressor is oxidative stress induced by FeCl₂ or CuCl₂ and a peroxide.

In yet another aspect, the invention provides a method of screening for modulators of TDP-43 aggregation comprising contacting a compound of Formula (I) with a cell that expresses TDP-43 and develops spontaneous inclusions.

In some embodiments, the stress granule comprises TDP-43, i.e., is a TDP-43 inclusion. Accordingly, in some embodiments, a compound of Formula (I) is a modulator of TDP-43 inclusions.

TDP-43 and other RNA-binding proteins function in both the nucleus and cytoplasm to process mRNA, e.g., by splicing mRNA, cleaving mRNA introns, cleaving untranslated regions of mRNA or modifying protein translation at the synapse, axon, dendrite or soma. Therefore, targeting other proteins that function in an analogous manner to TDP-43 or by processing mRNA may also be beneficial to prevent and treat neurodegeneration resulting from disease. For instance, the fragile X mental retardation 1 (FMRP) protein is essential for normal cognitive development (Nakamoto, M., et al. (2007) Proc Natl Acad Sci U.S.A. 104:15537-15542). The signaling systems that affect TDP-43 function might also affect this protein, thus improving cognitive function. This can be particularly important at the synapse where neurons communicate. Without wishing to be bound by a theory, the signaling systems that compounds of Formula (I) target may also modify these processes, which play a role in neurodegeneration or mental health illnesses (e.g., schizophrenia).

The cellular stress response follows a U-shaped curve. Overinduction of this pathway, such as observed in many neurodegenerative diseases, can be harmful for cells. However, a decreased stimulation of this pathway can also be harmful for cells, e.g., in the case of an acute stress, such as a stroke. Thus, the appropriate action for some diseases is the inhibition of stress granule formation, while for other diseases, stimulation of stress granule formation is beneficial.

In some embodiments, the TDP-43 protein in a stress granule may be wild-type or a mutant form of TDP-43. In some embodiments, the mutant form of TDP-43 comprises an amino acid addition, deletion, or substitution, e.g., relative to the wild type sequence of TDP-43. In some embodiments, the mutant form of TDP-43 comprises an amino acid substitution relative to the wild type sequence, e.g., a G294A, A135T, Q331K, or Q343R substitution. In some embodiments, the TDP-43 protein in a stress granule comprises a post-translational modification, e.g., phosphorylation of an amino acid side chain, e.g., T103, S104, S409, or S410. In some embodiments, post-translational modification of the TDP-43 protein in a stress granule may be modulated by treatment with a compound of the invention.

The table below shows the structures of exemplary compounds of the invention.

| Compound No. | Structure |
|---|---|
| 100 | 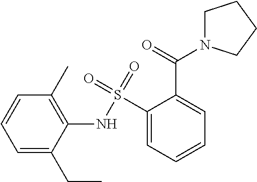 [SMILES: CCc1cccc(C)c1NS(=O)(=O)c1ccccc1C(=O)N1CCCC1] |
| 101 | 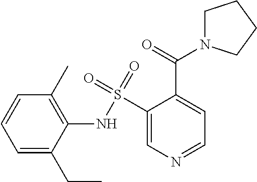 [SMILES: CCc1cccc(C)c1NS(=O)(=O)c1cnccc1C(=O)N1CCCC1] |
| 102 | 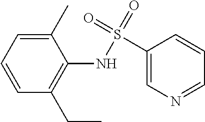 [SMILES: CCc1cccc(C)c1NS(=O)(=O)c1cccnc1] |

### Methods of Treatment

Neurodegenerative Diseases:

Without wishing to be bound by a theory, compounds of Formula (I) can be used to delay the progression of neurodegenerative illnesses where the pathology incorporates stress granules. Such illnesses include ALS and frontotemporal dementia, in which TDP-43 is the predominant protein that accumulates to form the pathology. This group also includes Alzheimer's disease and FTLD-U, where TDP-43 and other stress granule proteins co-localize with tau pathology. Because modulators of TDP-43 inclusions, such as compounds of Formula (I), can act to block the enzymes that signal stress granule formation (e.g., the three enzymes that phosphorylate eIF2a: PERK, GCN2 and HRI), compounds of Formula (I) may also reverse stress granules that might not include TDP-43. Accordingly, compounds of Formula (I) can be used for treatment of neurodegenerative diseases and disorders in which the pathology incorporates stress granules, such as Huntington's chorea and Creutzfeld-Jacob disease. Compounds of Formula (I) may also be used for treatment of neurodegenerative diseases and disorders that involve TDP-43 multisystem proteinopathy.

The term “neurodegenerative disease” as used herein, refers to a neurological disease characterized by loss or degeneration of neurons. The term “neurodegenerative disease” includes diseases caused by the involvement of genetic factors or the cell death (apoptosis) of neurons attributed to abnormal protein accumulation and so on. Additionally, neurodegenerative diseases include neurodegenerative movement disorders and neurodegenerative conditions relating to memory loss and/or dementia. Neurodegenerative diseases include tauopathies and α-synucleopathies. Exemplary neurodegenerative diseases include, but are not limited to, Alzheimer's disease, frontotemporal dementia (FTD), FTLD-U, FTD caused by mutations in the progranulin protein or tau protein (e.g., progranulin-deficient FTLD), frontotemporal dementia with inclusion body myopathy (IBMPFD), frontotemporal dementia with motor neuron disease, amyotrophic lateral sclerosis (ALS), amyotrophic lateral sclerosis with dementia (ALSD), Huntington's disease (HD), Huntington's chorea, prion diseases (e.g., Creutzfeld-Jacob disease, bovine spongiform encephalopathy, Kuru, or scrapie), Lewy Body disease, diffuse Lewy body disease (DLBD), polyglutamine (polyQ)-repeat diseases, trinucleotide repeat diseases, cerebral degenerative diseases, presenile dementia, senile dementia, Parkinsonism linked to chromosome 17 (FTDP-17), progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, HIV-associated dementia, Parkinson's disease, Parkinson's disease with dementia, dementia with Lewy bodies, Down's syndrome, multiple system atrophy, spinal muscular atrophy (SMA, e.g., SMA Type I (e.g., Werdnig-Hoffmann disease) SMA Type II, SMA Type III (e.g., Kugelberg-Welander disease), and congenital SMA with arthrogryposis), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, age-related disorders and dementias, Hallervorden-Spatz syndrome, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Schilder's disease, Batten disease, Cockayne syndrome, Kearns-Sayre syndrome, Gerstmann-Straussler-Scheinker syndrome, hereditary spastic paraparesis, Leigh's syndrome, demyelinating diseases, epilepsy, tremors, depression, mania, anxiety and anxiety disorders, sleep disorders (e.g., narcolepsy, fatal familial insomnia), acute brain injuries (e.g., stroke, head injury) and autism. As used herein, the term “α-synucleopathy” refers to a neurodegenerative disorder or disease involving aggregation of α-synuclein or abnormal α-synuclein in nerve cells in the brain (Ostrerova, N., et al. (1999) J Neurosci 19:5782:5791; Rideout, H. J., et al. (2004) J Biol Chem 279:46915-46920). α-Synucleopathies include, but are not limited to, Parkinson's disease, Parkinson's disease with dementia, dementia with Lewy bodies, Pick's disease, Down's syndrome, multiple system atrophy, amylotrophic lateral sclerosis (ALS), Hallervorden-Spatz syndrome, and the like.

As used herein, the term “tauopathy” refers to a neurodegenerative disease associated with the pathological aggregation of tau protein in the brain. Tauopathies include, but are not limited to, Alzheimer's disease, Pick's disease, corticobasal degeneration, Argyrophilic grain disease (AGD), progressive supranuclear palsy, Frontotemporal dementia, Frontotemporal lobar degeneration, or Pick's complex.

Musculoskeletal Diseases:

Musculoskeletal diseases and disorders as defined herein are conditions that affect the muscles, ligaments, tendons, and joints, as well as the skeletal structures that support them. Without wishing to be bound by a theory, aberrant expression of certain proteins, such as the full-length isoform of DUX4, has been shown to inhibit protein turnover and increase the expression and aggregation of cytotoxic proteins including insoluble TDP-43 in skeletal muscle cells (Homma, S. et al. Ann Clin Transl Neurol (2015) 2:151-166). As such, compounds of Formula (I) may be used to prevent or treat a musculoskeletal disease, e.g., a musculoskeletal disease that results in accumulation of TDP-43 and other stress granule proteins, e.g., in the nucleus, cytoplasm, or cell bodies of a muscle cell or motor neuron. Exemplary musculoskeletal diseases include muscular dystrophy, facioscapulohumeral muscular dystrophy (e.g., FSHD1 or FSHD2), Freidrich's ataxia, progressive muscular atrophy (PMA), mitochondrial encephalomyopathy (MELAS), multiple sclerosis, inclusion body myopathy, inclusion body myositis (e.g., sporadic inclusion body myositis), post-polio muscular atrophy (PPMA), motor neuron disease, myotonia, myotonic dystrophy, sacropenia, spasticity, multifocal motor neuropathy, inflammatory myopathies, paralysis, and other diseases or disorders relating to the aberrant expression of TDP-43 and altered proteostasis. In addition, compounds of Formula (I) may be used to prevent or treat symptoms caused by or relating to said musculoskeletal diseases, e.g., kyphosis, hypotonia, foot drop, motor dysfunctions, muscle weakness, muscle atrophy, neuron loss, muscle cramps, altered or aberrant gait, dystonias, astrocytosis (e.g., astrocytosis in the spinal cords), liver disease, inflammation, headache, pain (e.g., back pain, neck pain, leg pain, inflammatory pain), and the like. In some embodiments, a musculoskeletal disease or a symptom of a musculoskeletal disease may overlap with a neurodegenerative disease or a symptom of a neurodegenerative disease.

Cancers:

Cancer cells grow quickly and in low oxygen environments by activating different elements of the cellular stress response. Researchers have shown that drugs targeting different elements of the stress response can be anti-neoplastic. For example, rapamycin blocks mTOR, upregulates autophagy and inhibits some types of tumors. Proteasomal inhibitors, such as velcade (Millenium Pharma) are used to treat some cancers. HSP90 inhibitors, such as 17-allylaminogeldanamycin (17AAG), are currently in clinical trials for cancer. Without wishing to be bound by a theory, compounds of Formula (I) may also be used for treatment of cancer, as a greater understanding of the role of TDP-43 in RNA processing and transcription factor signaling has recently begun to emerge (Lagier-Tourenne, C., et al. (2010) Hum Mol Genet 19:R46-R64; Ayala, Y. M., et al. (2008) Proc Natl Acad Sci U.S.A. 105(10):3785-3789). Additionally, TDP-43 modulators can be combined with one or more cancer therapies, such as chemotherapy and radiation therapy.

A “cancer” in a subject refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may be a non-tumorigenic cancer cell, such as a leukemia cell. In some circumstances, cancer cells will be in the form of a tumor; such cells may exist locally within an animal, or circulate in the blood stream as independent cells, for example, leukemic cells. Examples of cancer include but are not limited to breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, ophthalmological cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, vulval cancer, and the like.

Other exemplary cancers include, but are not limited to, ACTH-producing tumors, acute lymphocytic leukemia, acute nonlymphocytic leukemia, cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, hairy cell leukemia, head & neck cancer, ophthalmological cancer, Hodgkin's lymphoma, Kaposi's sarcoma, kidney cancer, liver cancer, lung cancer (small and/or non-small cell), malignant peritoneal effusion, malignant pleural effusion, melanoma, mesothelioma, multiple myeloma, neuroblastoma, non-Hodgkin's lymphoma, osteosarcoma, ovarian cancer, ovary (germ cell) cancer, prostate cancer, pancreatic cancer, penile cancer, retinoblastoma, skin cancer, soft-tissue sarcoma, squamous cell carcinomas, stomach cancer, testicular cancer, thyroid cancer, trophoblastic neoplasms, uterine cancer, vaginal cancer, cancer of the vulva, Wilm's tumor, and the like.

Exemplary lymphomas include Hodgkin's lymphoma and non-Hodgkin's lymphoma. Further exemplification of non-Hodgkin's lymphoma include, but are not limited to, B-cell lymphomas (e.g., diffuse large B-cell lymphoma, primary mediastinal B-cell lymphoma, intravascular large B-cell lymphoma, follicular lymphoma, chronic lymphocytic leukemia/small lymphocytic lymphoma, mantle cell lymphoma, marginal zone B-cell lymphomas, extranodal marginal B-cell lymphomas, mucosa-associated lymphoid tissue (MALT) lymphomas, modal marginal zone B-cell lymphoma, splenic marginal zone B-cell lymphoma, Burkitt lymphoma, lymphoplasmacytic lymphoma, Waldenström's macroglobulinemia, hairy cell leukemia, and primary central nervous system (CNS) lymphoma) and T-cell lymphomas (e.g., precursor T-lymphoblastic lymphomalleukemia, peripheral T-cell lymphoma, cutaneous T-cell lymphoma, adult T-cell lymphoma (e.g., smoldering adult T-cell lymphoma, chronic adult T-cell lymphoma, acute adult T-cell lymphoma, lymphomatous adult T-cell lymphoma), angioimmunoblastic T-cell lymphoma, extranodal natural killer T-cell lymphoma nasal type (ENKL), enteropathy-associated intestinal T-cell lymphoma (EATL) (e.g., Type I EATL and Type II EATL), and anaplastic large cell lymphoma (ALCL)).

Ophthalmological Diseases:

Ophthalmological diseases and disorders (e.g., retinal diseases and disorders) as defined herein affect the retina and other parts of the eye and may contribute to impaired vision and blindness. Several ophthalmological diseases (e.g., retinal diseases) are characterized by the accumulation of protein inclusions and stress granules within or between cells of the eye, e.g., retinal cells and nearby tissues. In addition, an ophthalmological disease (e.g., retinal disease) may also be a symptom of or precursor to neurogenerative diseases, such as ALS and FTD (Ward, M. E., et al. (2014) J Exp Med 211(10):1937). Therefore, use of compounds that may inhibit formation of protein inclusions and stress granules, including compounds of Formula (I), may play an important role in the prevention or treatment of ophthalmological diseases (e.g., retinal diseases).

Exemplary ophthalmological diseases (e.g., retinal diseases) include, but are not limited to, macular degeneration (e.g., age-related macular degeneration), diabetes retinopathy, histoplasmosis, macular hole, macular pucker, Bietti's crystalline dystrophy, retinal detachment, retinal thinning, retinoblastoma, retinopathy of prematurity, Usher's syndrome, vitreous detachment, Refsum disease, retinitis pigmentosa, onchocerciasis, choroideremia, Leber congenital amaurosis, retinoschisis (e.g., juvenile retinoschisis), Stargardt disease, ophthalmoplegia, and the like.

Viral Infections:

Stress granules often form during viral illnesses, as viral infections often involve hijacking the cellular reproductive machinery toward production of viral proteins. In this case, inhibitors of stress granules can be useful for interfering with viral function. Other viruses appear to inhibit SG formation to prevent the cell from mobilizing a stress response. In such a case, an inducer of stress granules can interfere with viral activity and help combat viral infections (e.g., Salubrinal, a PERK inhibitor and stress granule inducer). Two viruses for which SG biology has been investigated include West Nile virus and respiratory syncytial virus (RSV) (Emara, M. E. and Brinton, M. A. (2007) Proc. Natl. Acad. Sci. USA 104(21): 9041-9046). Therefore, use of compounds that may inhibit formation of protein inclusions and stress granules, including compounds of Formula (I), may be useful for the prevention and/or treatment of a viral infection.

Exemplary viruses include, but are not limited to, West Nile virus, respiratory syncytial virus (RSV), Epstein-Barr virus (EBV), hepatitis A, B, C, and D viruses, herpes viruses, influenza viruses, chicken pox, avian flu viruses, smallpox, polio viruses, HIV, Ebola virus, and the like.

### Imaging

The compounds described herein are useful for detection and/or diagnosis of stress granules. Accordingly, they can be used as in vivo imaging agents of tissues and organs in various biomedical applications. When used in imaging applications, the compounds described herein typically comprise an imaging agent, which can be covalently or noncovalently attached to the compound.

As used herein, the term “imaging agent” refers to an element or functional group in a molecule that allows for the detection, imaging, and/or monitoring of the presence and/or progression of a condition(s), pathological disorder(s), and/or disease(s). The imaging agent may be an echogenic substance (either liquid or gas), non-metallic isotope, an optical reporter, a boron neutron absorber, a paramagnetic metal ion, a ferromagnetic metal, a gamma-emitting radioisotope, a positron-emitting radioisotope, or an x-ray absorber.

Suitable optical reporters include, but are not limited to, fluorescent reporters and chemiluminescent groups. A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound. Suitable fluorescent reporters include xanthene dyes, such as fluorescein or rhodamine dyes, including, but not limited to, Alexa Fluor® dyes (InvitrogenCorp.; Carlsbad, Calif.), fluorescein, fluorescein isothiocyanate (FITC), Oregon Green™, rhodamine, Texas red, tetrarhodamine isothiocynate (TRITC), 5-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N,N′-tetramefhyl-6-carboxyrhodamine (TAMRA), and 6-carboxy-X-rhodamine (ROX). Suitable fluorescent reporters also include the naphthylamine dyes that have an amino group in the alpha or beta position. For example, naphthylamino compounds include 1-dimethylamino-naphthyl-5-sulfonate, 1-anilino-8-naphthalene sulfonate, 2-p-toluidinyl-6-naphthalene sulfonate, and 5-(2′-aminoethyl)aminonaphthalene-1-sulfonic acid (EDANS). Other fluorescent reporter dyes include coumarins, such as 3-phenyl-7-isocyanatocoumarin; acridines, such as 9-isothiocyanatoacridine and acridine orange; N-(p(2-benzoxazolyl)phenyl)maleimide; cyanines, such as Cy2, indodicarbocyanine 3 (Cy3), indodicarbocyanine 5 (Cy5), indodicarbocyanine 5.5 (Cy5.5), 3-(-carboxy-pentyl)-3′ethyl-5,5′-dimethyloxacarbocyanine (CyA); 1H,5H,11H,15H-xantheno[2,3,4-ij:5,6,7-i′j′]diquinolizin-18-ium, 9-[2(or 4)-[[[6-[2,5-dioxo-1-pyrrolidinyl)oxy]-6-oxohexyl] amino]sulfonyl]-4(or 2)-sulfophenyl]-2,3,6,7,12,13,16,17-octahydro-inner salt (TR or Texas Red); BODIPY™ dyes; benzoxadiazoles; stilbenes; pyrenes; and the like. Many suitable forms of these fluorescent compounds are available and can be used as labels.

Examples of fluorescent proteins suitable for use as imaging agents include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566). Specific examples of GFP variants include, but are not limited to, enhanced GFP (EGFP), destabilized EGFP, the GFP variants described in Doan et al, (2005) Mol Microbiol 55:1767-1781, the GFP variant described in Crameri et al, (1996) Nat Biotechnol 14:315319, the cerulean fluorescent proteins described in Rizzo et al, (2004) Nat Biotechnol, 22:445 and Tsien, (1998) Annu Rev Biochem 67:509, and the yellow fluorescent protein described in Nagal et al, (2002) Nat Biotechnol 20:87-90. DsRed variants are described in, e.g., Shaner et al, (2004) Nat Biotechnol 22:1567-1572, and include mStrawberry, mCherry, mOrange, mBanana, mHoneydew, and mTangerine. Additional DsRed variants are described in, e.g., Wang et al, (2004) Proc Natl Acad Sci U.S.A 101:16745-16749, and include mRaspberry and mPlum. Further examples of DsRed variants include mRFPmars described in Fischer et al, (2004) FEBS Lett 577:227-232 and mRFPruby described in Fischer et al, (2006) FEBS Lett 580:2495-2502.

Suitable echogenic gases include, but are not limited to, a sulfur hexafluoride or perfluorocarbon gas, such as perfluoromethane, perfluoroethane, perfluoropropane, perfluorobutane, perfluorocyclobutane, perfluropentane, or perfluorohexane.

Suitable non-metallic isotopes include, but are not limited to, ¹¹C, ¹⁴C, ¹³N, ¹⁸F, ¹²³I, ¹²⁴I, and ¹²⁵I.

Suitable radioisotopes include, but are not limited to, ⁹⁹mTc, ⁹⁵Tc, ¹¹¹In, ⁶²Cu, ⁶Cu, Ga, 68Ga, and 153Gd.

Suitable paramagnetic metal ions include, but are not limited to, Gd(III), Dy(III), Fe(III), and Mn(II).

Suitable X-ray absorbers include, but are not limited to, Re, Sm, Ho, Lu, Pm, Y, Bi, Pd, Gd, La, Au, Au, Yb, Dy, Cu, Rh, Ag, and Ir.

In some embodiments, the radionuclide is bound to a chelating agent or chelating agent-linker attached to the aggregate. Suitable radionuclides for direct conjugation include, without limitation, ¹⁸F, ¹²⁴I, ¹²⁵I, ¹³¹I, and mixtures thereof. Suitable radionuclides for use with a chelating agent include, without limitation, ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and mixtures thereof. Suitable chelating agents include, but are not limited to, DOTA, BAD, TETA, DTPA, EDTA, NTA, HDTA, their phosphonate analogs, and mixtures thereof. One of skill in the art will be familiar with methods for attaching radionuclides, chelating agents, and chelating agent-linkers to the aggregate or small molecule.

A detectable response generally refers to a change in, or occurrence of, a signal that is detectable either by observation or instrumentally. In certain instances, the detectable response is fluorescence or a change in fluorescence, e.g., a change in fluorescence intensity, fluorescence excitation or emission wavelength distribution, fluorescence lifetime, and/or fluorescence polarization. One of skill in the art will appreciate that the degree and/or location of labeling in a subject or sample can be compared to a standard or control (e.g., healthy tissue or organ). In certain other instances, the detectable response the detectable response is radioactivity (i.e., radiation), including alpha particles, beta particles, nucleons, electrons, positrons, neutrinos, and gamma rays emitted by a radioactive substance such as a radionuclide.

Specific devices or methods known in the art for the in vivo detection of fluorescence, e.g., from fluorophores or fluorescent proteins, include, but are not limited to, in vivo near-infrared fluorescence (see, e.g., Frangioni, (2003) Curr Opin Chem Biol 7:626-634), the Maestro™ in vivo fluorescence imaging system (Cambridge Research & Instrumentation, Inc.; Woburn, Mass.), in vivo fluorescence imaging using a flying-spot scanner (see, e.g., Ramanujam et al, (2001) IEEE Transactions on Biomedical Engineering, 48:1034-1041, Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, or signal amplification using photomultiplier tubes.

Any device or method known in the art for detecting the radioactive emissions of radionuclides in a subject is suitable for use in the present invention. For example, methods such as Single Photon Emission Computerized Tomography (SPECT), which detects the radiation from a single photon gamma-emitting radionuclide using a rotating gamma camera, and radionuclide scintigraphy, which obtains an image or series of sequential images of the distribution of a radionuclide in tissues, organs, or body systems using a scintillation gamma camera, may be used for detecting the radiation emitted from a radiolabeled aggregate. Positron emission tomography (PET) is another suitable technique for detecting radiation in a subject.

Magnetic resonance imaging (MRI), nuclear magnetic resonance imaging (NMRI), or magnetic resonance tomography (MRT) is a medical imaging technique used in radiology to visualize detailed internal structures. MRI makes use of the property of nuclear magnetic resonance (NMR) to image nuclei of atoms inside the body. Thus, labels having magnetic properties can be detected using MRI and/or related technologies.

SG proteins, such as TDP-43, undergo translocation to the cytoplasm and may form aggregates. Translocation likely requires a post-translational modification as well as binding to a transport protein. Aggregation is often associated with a change in protein conformation. Modulators of TDP-43 can bind to SG proteins specifically under states of cytoplasmic translocation (for instance, because they recognize a binding site enabled by a post-translational modification) or SG proteins that are in an aggregated state associated with SGs. Thus, modulators of TDP-43 inclusions can be used to image areas in a subject's body that have increased levels of SGs, either physiological or pathological. For instance, in ALS and Alzheimer's disease, the inventors have demonstrated that TDP-43 associates with the pathological form of TDP-43 that accumulates. Thus, compounds that recognize aggregated TDP-43 can be used to image pathology, much like the imaging agent PiB, which is currently used in Alzheimer's research. However, a drawback to use of PiB in imaging protein aggregates is that it recognizes amyloid protein, which accumulates both in patients with Alzheimer's disease and in many non-affected people. However, an agent that recognizes SGs would specifically target patients that have demonstrated intracellular pathology, such as neurofibrillary tangles, which are associated with SGs. Such agents can be used to diagnose patients at risk of developing a neurodegenerative illness.

Additionally, imaging of SGs in a subject can be used to localize pain. For example, a compound of Formula (I) can be administered to a subject experiencing pain, wherein the pain is difficult to localize. Subsequent imaging may be used to localize the area of the body exhibiting this pain, revealing disease or injury. This can greatly speed diagnosis and can be generally applicable throughout the medical arts.

Further, the compounds described herein can be used to image organs for transplants. Organs are harvested for transplants, such as kidneys and hearts. A problem in the field is that it is unclear to medical professionals how well the organ survived the harvesting and transport to the receiving hospital. Sometimes, organs are transplanted only to have them fail because they were injured in transport. A quick cytologic stain with a stress granule marker would represent a large advance for the field. Accordingly, compound of Formula (I) may be used as in the analysis of organs for transplantation.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein, the terms “compounds” and “agent” are used interchangeably to refer to the inhibitors/antagonists/agonists of the invention. In certain embodiments, the compounds are small organic or inorganic molecules, e.g., with molecular weights less than 7500 amu, preferably less than 5000 amu, and even more preferably less than 2000, 1500, 1000, 750, 600, or 500 amu. In certain embodiments, one class of small organic or inorganic molecules are non-peptidyl, e.g., containing 2, 1, or no peptide and/or saccharide linkages.

As used herein the term “comprising” or “comprises” is used in reference to compositions, methods, and respective component(s) thereof that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Unless otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” refer to one or to more than one, unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction”, “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase”, “enhance” or “activate” are all used herein to generally mean an increase by a statically significant amount; for the avoidance of any doubt, the terms “increased”, “increase”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, intrathecal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the compositions are administered by intravenous infusion or injection.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, at least one symptom of a disease or disorder is alleviated by at least 5%, at least 10%, at least 20%, at least 30%, at least 40%, or at least 50%.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. “Less effective” means that the treatment results in a therapeutically significant lower level of pharmacological effectiveness and/or a therapeutically greater level of adverse physiological effects.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, an amount of a compound or combination effective to treat a disorder (e.g., a disorder as described herein), “therapeutically effective amount”, “effective amount” or “effective course” refers to an amount of the compound or combination which is effective, upon single or multiple dose administration(s) to a subject, in treating a subject, or in curing, alleviating, relieving or improving a subject with a disorder (e.g., a disorder as described herein) beyond that expected in the absence of such treatment. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. The terms, “patient” and “subject” are used interchangeably herein. The term “nucleic acid” as used herein refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

As used herein, the terms “modulator of stress granule” and “stress granule modulator” refer to compounds and compositions of Formula (I) that modulate the formation and/or disaggregation of stress granules.

The term “TDP-43 inclusion” as used herein refers to protein-mRNA aggregates that comprise a TDP-43 protein. The TDP-43 protein in a stress granule can be wild-type or a mutant form of TDP-43.

As used herein, the terms “modulator of TDP-43 inclusion” and “TDP-43 inclusion modulator” refer to compounds and compositions of Formula (I) that modulate the formation and/or disaggregation of cytoplasmic TDP-43 inclusions.

### Chemical Definitions

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, propyl, butyl, and pentyl.

For compounds of the invention in which a variable appears more than once, each variable can be a different moiety selected from the Markush group defining the variable. For example, where a structure is described having two R groups that are simultaneously present on the same compound; the two R groups can represent different moieties selected from the Markush group defined for R.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

If a compound of the present invention is depicted in the form of a chemical name and as a formula, in case of any discrepancy, the formula shall prevail.

The symbol [symbol] , whether utilized as a bond or displayed perpendicular to a bond indicates the point at which the displayed moiety is attached to the remainder of the molecule, solid support, etc.

As used herein, “alkyl,” by itself or as part of another substituent, means, unless otherwise stated, a straight or branched chain, and can have a number of carbon atoms optionally designated (i.e., C₁-C₆ means one to six carbons). Examples of saturated hydrocarbon groups include, but are not limited to, groups such as methyl, ethyl, n-propyl, isopropyl, n-butyl, t-butyl, isobutyl, sec-butyl, n-pentyl, isopentyl, homologs and isomers of, for example, n-pentyl, n-hexyl, and the like.

As used herein, “alkenyl” can be a straight or branched hydrocarbon chain, containing at least one double bond, and having from two to six carbon atoms (i.e., C₂-C₆ alkenyl). Examples of alkenyl groups, include, but are not limited to, groups such as ethenyl (i.e., vinyl), prop-1-enyl (i.e., allyl), but-1-enyl, pent-1-enyl, penta-1,4-dienyl, and the like.

As used herein, “alkoxy” can be a straight chain or branched alkoxy group (e.g., C₁-C₆ alkyl-O—) having from one to six carbon atoms (i.e., C₁-C₆ alkoxy). Examples of alkoxy groups, include, but are not limited to, groups such as methoxy, ethoxy, propyloxy, isopropyloxy, butyloxy, isobutyloxy, tert-butyloxy, pentyloxy, or hexyloxy, and the like.

As used herein, “alkynyl” can be a straight or branched hydrocarbon chain, containing at least one triple bond, having from two to six carbon atoms (i.e., C₂-C₆ alkynyl). Examples of alkynyl groups, include, but are not limited to, groups such as ethynyl, propynyl, butynyl, pentynyl, hexynyl, and the like.

The term “acyl” as used herein refers to a group represented by the general formula hydrocarbylC(O)—, preferably alkylC(O)—.

Herein, the term “aliphatic group” refers to a straight-chain, branched-chain, or cyclic aliphatic hydrocarbon group and includes saturated and unsaturated aliphatic groups, such as an alkyl group, an alkenyl group, and an alkynyl group.

As used herein, “alkylthio” refers to an alkyl group, as defined above, having a sulfur radical attached thereto. Non-limiting examples of alkylthio groups include —S-alkyl (e.g., methylthio, ethylthio, etc.), —S-alkenyl, —S-alkynyl, and the like.

As used herein, “amide” or “amido” refers to a chemical moiety with the formula —C(O)NR^{a}— or —NR^{a}C(O)— wherein R^{a} is H or C₁-C₆ alkyl.

As used herein, “amino” or “amine” refers to a —NH₂ radical group.

As used herein, “substituted amino” refers to an amino group of the formula —N(R¹⁰)₂, wherein at least one of R¹⁰ is not a hydrogen. The terms “alkylamino” and “dialkylamino” refer to (alkyl)ₓ(amino)-, wherein x is 1 or 2 and the alkyl and amino moieties are as disclosed herein. The term “arylamino” refers to aryl(amino)-, wherein the aryl and amino groups are as disclosed herein.

As used herein, “aryl” refers to a polyunsaturated, aromatic, hydrocarbon moiety which can be a single ring or multiple rings (e.g., 1 to 2 rings) which are fused together or linked covalently, having from six to twelve carbon atoms (i.e. C₆-C₁₂ aryl). Non-limiting examples of aryl groups include phenyl, 1-naphthyl, 2-naphthyl, and 4-biphenyl and the like.

As used herein, “arylalkyl” refers to an (aryl)alkyl-radical wherein aryl and alkyl moieties are as disclosed herein.

As used herein, “aryloxy” refers to —O-(aryl), wherein the aryl moiety is as defined herein.

As used herein, “arylalkoxy” refers to —O-(arylalkyl), wherein the arylalkyl moiety is as defined herein.

As used herein, “cyano” refers to a —CN radical.

As used herein, “cycloalkyl” refers to a monocyclic or polycyclic radical that contains only carbon and hydrogen, and may be saturated, or partially unsaturated. Cycloalkyl groups include groups having from 3 to 10 ring atoms (i.e. C₃-C₁₀ cycloalkyl). Examples of cycloalkyl groups include, but are not limited to, groups such as cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like.

As used herein, “cycloalkylalkyl” as used herein refers to a -(cycloalkyl)-alkyl radical where cycloalkyl and alkyl are as defined herein. As used herein, “halo” or “halogen,” independently or as part of another substituent, mean, unless otherwise stated, a fluorine, chlorine, bromine, or iodine atom. The term “halide” by itself or as part of another substituent refers to a fluoride, chloride, bromide, or iodide atom.

As used herein, “haloalkyl” and “haloalkoxy” can include alkyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine (e.g., —C₁-C₆ alkyl-CF₃, —C₁-C₆ alkyl-C₂F). Non-limiting examples of haloalkyl include trifluoroethyl, trifluoropropyl, trifluoromethyl, fluoromethyl, diflurormethyl, and fluroisopropyl.

As used herein, “heteroalkyl” refers to alkyl structures wherein one or more of the carbon atoms in the structure is replaced or substituted with a heteroatom, wherein the heteroatoms are selected from nitrogen, oxygen, sulfur, and phosphorus.

As used herein, “heteroaryl” refers to a 5- to 14-membered aromatic radical (e.g., C₂-C₁₃ heteroaryl) that includes one or more ring heteroatoms selected from nitrogen, oxygen and sulfur, and which may be a monocyclic or bicyclic ring system. The polycyclic heteroaryl group may be fused or non-fused. The heteroatom(s) in the heteroaryl radical is optionally oxidized. One or more nitrogen atoms, if present, are optionally quaternized. The heteroaryl is attached to the rest of the molecule through any atom of the ring(s). The term “heteroaryl” is intended to include all the possible isomeric forms. Examples of heteroaryl groups include without limitation, pyridyl, pyrimidinyl, pyrazinyl, pyridazinyl, triazinyl, furanyl, quinolyl, isoquinolyl, thienyl, imidazolyl, thiazolyl, indolyl, pyrrolyl, oxazolyl, oxadiazolyl, benzofuryl, benzothienyl, benzthiazolyl, isoxazolyl, pyrazolyl, triazolyl, tetrazolyl, indazolyl, thiadiazolyl, isothiazolyl, benzothienyl, purinyl, carbazolyl, benzimidazolyl, indolinyl, and the like.

As used herein, “heterocyclyl” can be a stable 3- to 18-membered non-aromatic mono, di, or tricyclic ring radical that comprises two to twelve carbon atoms and from one to six heteroatoms selected from nitrogen, oxygen and sulfur. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Examples of heterocyclyl groups include, but are not limited to, groups such as dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, azetidinyl, azabicyclohexyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, lactones, lactams, and the like.

As used herein, “heteroarylalkyl” refers to refers to an (heteroaryl)alkyl-radical wherein the heteroaryl and alkyl moieties are as disclosed herein.

As used herein, “heteraryloxy” refers to —O-(heteroaryl), wherein the heteroaryl moiety is as defined herein.

As used herein, “heterocycloalkyl” refers to an (heterocyclyl)alkylradical wherein the heterocyclyl and alkyl moieties are as disclosed herein. Examples of heterocycloalkyl groups include, but are not limited to, groups such as dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, and the like covalently bonded to one or more alkyl moieties as defined herein.

As used herein, “hydroxy” or “hydroxyl” refers to —OH.

As used herein, “nitro” refers to —NO₂.

As used herein, “keto” refers to —C═O.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

Many of the terms given above may be used repeatedly in the definition of a formula or group and in each case have one of the meanings given above, independently of one another.

As used herein, the term “substituted” or “substituted with” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds (e.g., alkyl, alkenyl, alkynyl, cycloalkyl, heterocycloalkyl, aryl, or heteroaryl, any of which may itself be further substituted), as well as halogen, carbonyl (e.g., aldehyde, ketone, ester, carboxyl, or formyl), thiocarbonyl (e.g., thioester, thiocarboxylate, or thioformate), amino, —N(R^{b})(R^{c}), wherein each R^{b} and R^{c} is independently H or C₁-C₆ alkyl, cyano, nitro, —SO₂N(R^{b})(R^{c}), —SOR^{d}, and S(O)₂R^{d}, wherein each R^{b}, R^{c}, and R^{d} is independently H or C₁-C₆ alkyl. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

Contemplated equivalents of the compounds described above include compounds which otherwise correspond thereto, and which have the same general properties thereof (e.g., the ability to inhibit the formation of TDP-43 inclusions), wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

Certain compounds of the present invention may exist in particular geometric or stereoisomeric forms. The present invention contemplates all such compounds, including cis- and trans-isomers, R- and S-enantiomers, diastereomers, (d)-isomers, (l)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Methods of preparing substantially isomerically pure compounds are known in the art. If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts may be formed with an appropriate optically active acid or base, followed by resolution of the diastereomers thus formed by fractional crystallization or chromatographic means well known in the art, and subsequent recovery of the pure enantiomers. Alternatively, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Techniques for inverting or leaving unchanged a particular stereocenter, and those for resolving mixtures of stereoisomers are well known in the art, and it is well within the ability of one of skill in the art to choose an appropriate method for a particular situation. See, generally, Furniss et al. (eds.), Vogel's Encyclopedia of Practical Organic Chemistry 5^{th} Ed., Longman Scientific and Technical Ltd., Essex, 1991, pp. 809-816; and Heller, (1990) Acc Chem Res 23:128.

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer.

ee=(90−10)/100=80%.

Thus, a composition containing 90% of one enantiomer and 10% of the other enantiomer is said to have an enantiomeric excess of 80%.

The “diastereomeric excess” or “% diastereomeric excess” of a composition can be calculated using the equation shown below. In the example shown below a composition contains 90% of one diastereomer, and 10% of another enantiomer.

ee=(90−10)/100=80%.

Thus, a composition containing 90% of one diastereomer and 10% of the other diastereomer is said to have a diastereomeric excess of 80%.

In addition, compounds of Formula (I) can include one or more isotopes of the atoms present in Formula (I). For example, compounds of Formula (I) can include: those in which H (or hydrogen) is replaced with any isotopic form of hydrogen including ¹H, ²H or D (Deuterium), and ³H (Tritium); those in which C is replaced with any isotopic form of carbon including ¹²C, ¹³C, and ¹⁴C; those in which O is replaced with any isotopic form of oxygen including ¹⁶O, ¹⁷O and ¹⁸O; those in which N is replaced with any isotopic form of nitrogen including ¹³N, ¹⁴N and ¹⁵N; those in which P is replaced with any isotopic form of phosphorous including ³¹P and ³²P; those in which S is replaced with any isotopic form of sulfur including ³²S and ³⁵S; those in which F is replaced with any isotopic form of fluorine including ¹⁹F and ¹⁸F; and the like. In an embodiment, compounds represented by Formula (I) comprise isomers of the atoms therein in their naturally occurring abundance.

Certain compounds disclosed herein can exist in unsolvated forms as well as solvated forms, including hydrated forms. In general, the solvated forms are equivalent to unsolvated forms and are encompassed within the scope of the present invention. Certain compounds disclosed herein may exist in multiple crystalline or amorphous forms. In general, all physical forms are equivalent for the uses contemplated by the present invention and are intended to be within the scope of the present invention.

Contemplated equivalents of the compounds described above include compounds which otherwise correspond thereto, and which have the same general properties thereof (e.g., the ability to inhibit TDP-43 inclusions), wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, trifluoroacetic, propionic, isobutyric, maleic, malonic, benzoic, succinic, suberic, fumaric, lactic, mandelic, phthalic, benzensulfonic, p-tolylsulfonic, citric, tartaric, methanesulfonic, and the like. Also included are the salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The term “low enough pyrogen activity”, with reference to a pharmaceutical preparation, refers to a preparation that does not contain a pyrogen in an amount that would lead to an adverse effect (e.g., irritation, fever, inflammation, diarrhea, respiratory distress, endotoxic shock, etc.) in a subject to which the preparation has been administered. For example, the term is meant to encompass preparations that are free of, or substantially free of, an endotoxin such as, for example, a lipopolysaccharide (LPS).[0001] The designations or prefixes “(+) and (−)” are employed to designate the sign of rotation of plane-polarized light by the compound, with (−) meaning that the compound is levorotatory (rotates to the left). A compound prefixed with (+) is dextrorotatory (rotates to the right).

The term “racemic mixture,” “racemic compound” or “racemate” refers to a mixture of the two enantiomers of one compound. An ideal racemic mixture is one wherein there is a 50:50 mixture of both enantiomers of a compound such that the optical rotation of the (+) enantiomer cancels out the optical rotation of the (−) enantiomer.

The term “resolving” or “resolution” when used in reference to a racemic mixture refers to the separation of a racemate into its two enantiomorphic forms (i.e., (+) and (−); 65 (R) and (S) forms). The terms can also refer to enantioselective conversion of one isomer of a racemate to a product.

The term “analog” as used herein refers to a compound that results from substitution, replacement or deletion of various organic groups or hydrogen atoms from a parent compound. As such, some monoterpenoids can be considered to be analogs of monoterpenes, or in some cases, analogs of other monoterpenoids, including derivatives of monoterpenes. An analog is structurally similar to the parent compound, but can differ by even a single element of the same valence and group of the periodic table as the element it replaces.

The term “derivative” as used herein refers to a chemical substance related structurally to another, i.e., an “original” substance, which can be referred to as a “parent” compound. A “derivative” can be made from the structurally-related parent compound in one or more steps. The phrase “closely related derivative” means a derivative whose molecular weight does not exceed the weight of the parent compound by more than 50%. The general physical and chemical properties of a closely related derivative are also similar to the parent compound.

As used herein, a “prodrug” refers to compounds that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject (e.g., an ester), but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. For examples, see Bioreversible Carriers in Drug in Drug Design, Theory and Application, E. B. Roche, ed., APHA Acad Pharm Sci (1987); Design of Prodrugs, H. Bundgaard, Elsevier (1985); Gaignault et al. (1996) Pract Med Chem 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., (1990) Eur J Drug Metab Pharmacokinet 15(2):143-53; Bundgaard, (1979) Arch Pharm Chemi 86(1): 1-39; Bundgaard H. (1987) Controlled Drug Delivery 17: 179-96 (1987); Fleisher et al. (1996) Drug Delivery Rev 19(2):115-130; Fleisher et al. (1985) Methods Enzymol 112:360-81 Sinhababu and Thakker, (1996) Adv Drug Delivery Rev 19(2):241-273; Tan et al. (1999) Adv Drug Delivery Rev 39(1-3):117-151; Taylor, (1996) Adv Drug Delivery Rev 19(2):131-148; Wiebe and Knaus, (1999) Adv Drug Delivery Rev 39(1-3):63-80; Waller et al., (1989) Br J Clin Pharmac 28: 497-507.

### Pharmaceutical Compositions and Routes of Administration

Pharmaceutical compositions containing compounds described herein such as a compound of Formula (I) or pharmaceutically acceptable salt thereof can be used to treat or ameliorate a disorder described herein, for example, a neurodegenerative disease, a cancer, an ophthalmological disease (e.g., a retinal disease), or a viral infection.

The amount and concentration of compounds of Formula (I) in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

While it is possible for a compound of the present invention to be administered alone, it is preferable to administer the compound as a pharmaceutical formulation (composition), where the compound is combined with one or more pharmaceutically acceptable diluents, excipients or carriers. The compounds according to the invention may be formulated for administration in any convenient way for use in human or veterinary medicine. In certain embodiments, the compound included in the pharmaceutical preparation may be active itself, or may be a prodrug, e.g., capable of being converted to an active compound in a physiological setting. Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Thus, another aspect of the present invention provides pharmaceutically acceptable compositions comprising a therapeutically effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; (9) nasally; or (10) intrathecally. Additionally, compounds can be implanted into a patient or injected using a drug delivery system. See, for example, Urquhart, et al., (1994) Ann Rev Pharmacol Toxicol 24:199-236; Lewis, ed. “Controlled Release of Pesticides and Pharmaceuticals” (Plenum Press, New York, 1981); U.S. Pat. No. 3,773,919; and U.S. Pat. No. 35 3,270,960.

The phrase “therapeutically effective amount” as used herein means that amount of a compound, material, or composition comprising a compound of the present invention which is effective for producing some desired therapeutic effect, e.g., by inhibiting TDP-43 inclusions, in at least a sub-population of cells in an animal and thereby blocking the biological consequences of that function in the treated cells, at a reasonable benefit/risk ratio applicable to any medical treatment.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) cyclodextrins such as Captisol®; and (22) other non-toxic compatible substances employed in pharmaceutical formulations.

As set out above, certain embodiments of the present compounds may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (see, for example, Berge et al. (1977) “Pharmaceutical Salts”, J Pharm Sci 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal, vaginal, or urethral administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Alternatively or additionally, compositions can be formulated for delivery via a catheter, stent, wire, or other intraluminal device. Delivery via such devices may be especially useful for delivery to the heart, lung, bladder, urethra, ureter, rectum, or intestine. Furthermore, compositions can be formulated for delivery via a dialysis port.

Ophthalmic formulations, eye ointments, powders, solutions and the like, are also contemplated as being within the scope of this invention.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound of the invention to animal feed is preferably accomplished by preparing an appropriate feed premix containing the active compound in an effective amount and incorporating the premix into the complete ration. Alternatively, an intermediate concentrate or feed supplement containing the active ingredient can be blended into the feed. The way in which such feed premixes and complete rations can be prepared and administered are described in reference books (such as “Applied Animal Nutrition”, W.H. Freedman and CO., San Francisco, U.S.A., 1969 or “Livestock Feeds and Feeding” O and B books, Corvallis, Oreg., U.S.A., 1977).

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with neurodegenerative disease or disorder, cancer, or viral infections.

In addition, the methods described herein can be used to treat domesticated animals and/or pets. A subject can be male or female. A subject can be one who has been previously diagnosed with or identified as suffering from or having a neurodegenerative disease or disorder, a disease or disorder associated with cancer, a disease or disorder associated with viral infection, or one or more complications related to such diseases or disorders but need not have already undergone treatment.

### Dosages

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

The compound and the pharmaceutically active agent can be administrated to the subject in the same pharmaceutical composition or in different pharmaceutical compositions (at the same time or at different times). When administrated at different times, the compound and the pharmaceutically active agent can be administered within 5 minutes, 10 minutes, 20 minutes, 60 minutes, 2 hours, 3 hours, 4, hours, 8 hours, 12 hours, 24 hours of administration of the other agent. When the inhibitor and the pharmaceutically active agent are administered in different pharmaceutical compositions, routes of administration can be different.

The amount of compound that can be combined with a carrier material to produce a single dosage form will generally be that amount of the inhibitor that produces a therapeutic effect. Generally out of one hundred percent, this amount will range from about 0.1% to 99% of inhibitor, preferably from about 5% to about 70%, most preferably from 10% to about 30%.

Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC₅₀ (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

The dosage may be determined by a physician and adjusted, as necessary, to suit observed effects of the treatment. Generally, the compositions are administered so that the compound of Formula (I) is given at a dose from 1 ng/kg to 200 mg/kg, 10 ng/kg to 100 mg/kg, 10 ng/kg to 50 mg/kg, 100 ng/kg to 20 mg/kg, 100 ng/kg to 10 mg/kg, 100 ng/kg to 1 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 10 μg/kg to 10 mg/kg, 10 μg/kg to 50 mg/kg, 10 mg/kg to 20 mg/kg, 10 μg/kg to 10 mg/kg, 10 μg/kg to 1 mg/kg, 100 μg/kg to 50 mg/kg, 100 μg/kg to 20 mg/kg, 1 mg/kg to 100 mg/kg, 1 mg/kg to 50 mg/kg, 1 mg/kg to 20 mg/kg, 1 mg/kg to 10 mg/kg, 1 μg/kg to 10 mg/kg, 10 mg/kg to 100 mg/kg, 10 mg/kg to 50 mg/kg, 10 mg/kg to 20 mg/kg, or 50 mg/kg to 100 mg/kg. It is to be understood that ranges given here include all intermediate ranges, e.g., the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. It is to be further understood that the ranges intermediate to the given above are also within the scope of this invention, for example, in the range 1 mg/kg to 10 mg/kg, dose ranges such as 2 mg/kg to 8 mg/kg, 3 mg/kg to 7 mg/kg, 4 mg/kg to 6 mg/kg, and the like.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the drugs. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

The present invention contemplates formulation of the subject compounds in any of the aforementioned pharmaceutical compositions and preparations. Furthermore, the present invention contemplates administration via any of the foregoing routes of administration. One of skill in the art can select the appropriate formulation and route of administration based on the condition being treated and the overall health, age, and size of the patient being treated.

### EXAMPLES

Examples are provided below to facilitate a more complete understanding of the invention. The following examples illustrate exemplary modes of making and practicing the invention. However, the scope of the invention is not limited to specific embodiments disclosed in these Examples, which are for purposes of illustration only, since alternative methods can be utilized to obtain similar results.

### Example 1. Synthesis of N-(2-ethyl-6-methylphenyl)-2-(pyrrolidine-1-carbonyl)-benzenesulfonamide (Compound 100)

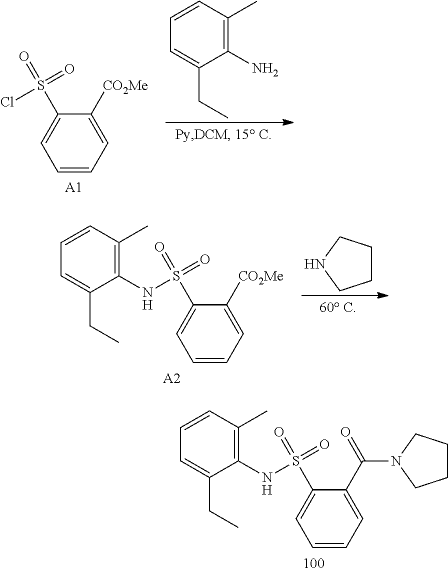

### Step 1: Methyl 2-(N-(2-ethyl-6-methylphenyl)sulfamoyl)benzoate (A2)

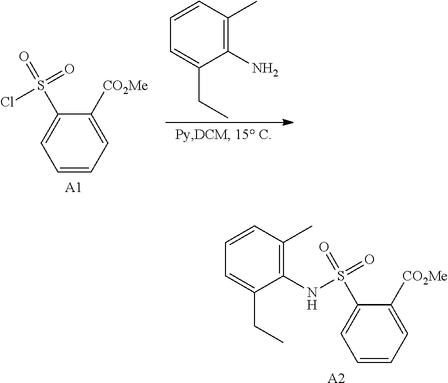

To a solution of A1 (300 mg, 1.28 mmol) and 2-ethyl-6-methyl-aniline (173 mg, 1.28 mmol) in DCM (5.00 mL) was added pyridine (202 mg, 2.56 mmol). The mixture was stirred at 40° C. for 16 hrs, after which TLC analysis (petroleum ether:ethyl acetate=5:1, R_{f}=0.29) indicated the reaction was complete. The reaction mixture was concentrated under reduced pressure to remove solvent to afford A2 (300 mg, crude) as a red oil, which was directly used without further purification.

### Step 2: N-(2-ethyl-6-methylphenyl)-2-(pyrrolidine-1-carbonyl)-benzenesulfonamide (Compound 100)

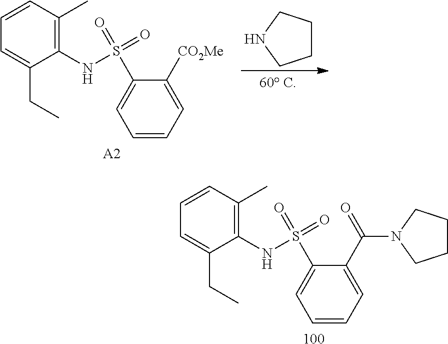

A mixture of A2 (300 mg, 900 umol) and pyrrolidine (1.02 g, 14.3 mmol) was stirred at 60° C. for 3 hrs, after which TLC analysis (petroleum ether:ethyl acetate=5:1, R_{f}=0.15) indicated A2 was consumed completely. The reaction mixture was concentrated under reduced pressure to give a residue, which was purified by prep-HPLC (neutral condition) to provide the title compound (90 mg, 26.0% yield) as a yellow solid. ¹H-NMR (400 MHz CDCl₃): δ 7.71-7.73 (m, 2H), 7.44-7.58 (m, 1H), 7.41-7.43 (m, 2H), 7.08-7.42 (m, 2H), 7.05 (d, J=10.4 Hz, 1H), 3.72 (br. s., 2H), 3.29 (t, J=6.8 Hz, 2H), 2.52 (br. s., 2H), 2.16 (s, 3H), 1.90-2.05 (m, 4H), 1.05 (t, J=7.6 Hz, 3H).

### Example 2. Synthesis of N-(2-ethyl-6-methylphenyl)-4-(pyrrolidine-1-carbonyl)pyridine-3-sulfonamide (Compound 101)

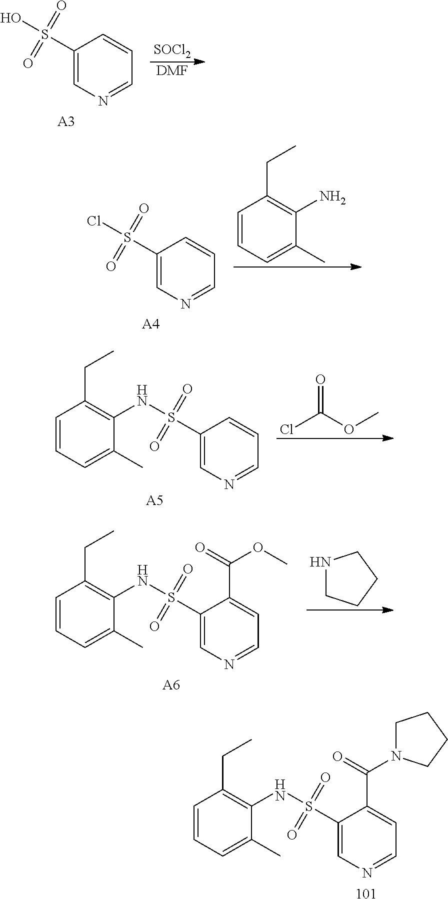

### Step 1: Pyridine-3-sulfonyl chloride (A4)

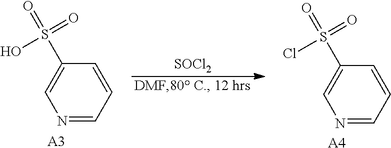

To a mixture of A3 (9.00 g, 56.6 mmol) in SOCl₂ (108.00 mL) was added DMF (5.00 mL) in one portion at 25° C. under N₂. The mixture heated to 80° C. and stirred for 12 hours, after which LCMS analysis indicated the reaction was complete. The reaction mixture was concentrated under reduced pressure to give A4 (9.00 g, yield: 89.6%), which was used in the next step without further purification. ¹H NMR (400 MHz, CDCl₃) δ:8.98 (s, 1H), 8.91 (d, J=5.6 Hz, 1H), 8.72 (d, J=8.0 Hz, 1H), 8.07-8.10 (m, 1H).

### Step 2: N-(2-ethyl-6-methylphenyl)pyridine-3-sulfonamide (Compound 102)

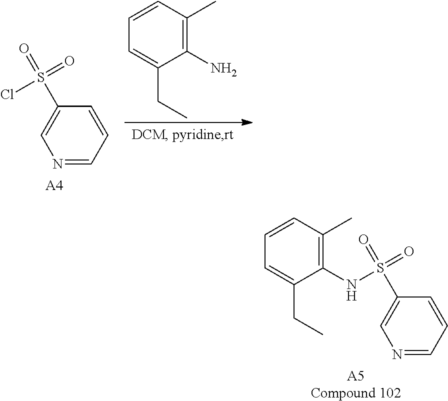

To a solution of 2-ethyl-6-methylaniline (4.57 g, 33.9 mmol) in CH₂Cl₂ (100 mL) and pyridine (20.0 mL) was added a solution of A4 (5.00 g, 28.2 mmol) in CH₂Cl₂ (50.00 mL) and pyridine (30.0 mL) dropwise at 0° C. under N₂. The mixture was stirred at 25° C. for 2 hours, at which point LCMS analysis indicated the reaction was complete. The reaction mixture was concentrated under reduced pressure to remove CH₂Cl₂ and pyridine, and the residue was purified by column chromatography (petroleum ether/ethyl acetate=1/1, R_{f}=0.43) to give A5 (5.30 g, 68.1% yield) as a light yellow solid. ¹H NMR (400 MHz, CDCl₃) δ: 8.87 (s, 1H), 8.74 (d, J=3.6 Hz, 1H), 7.91 (d, J=7.6 Hz, 1H), 7.33-7.35 (m, 1H), 7.10 (d, J=7.2 Hz, 1H), 6.96-7.03 (m, 2H), 6.20 (br. s., 1H), 2.34-2.39 (m, 2H), 1.99 (s, 3H), 0.94-1.00 (s, 3H).

### Step 3: 2-ethyl-6-methylaniline (A6)

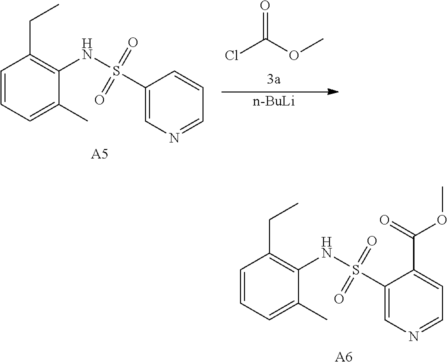

To a solution of A5 (1.00 g, 3.62 mmol) in THF (20.0 mL) was slowly added n-BuLi (579 mg, 9.05 mmol) under N₂ at −78° C. The reaction temperature was raised to −20° C. and cooled down to −78° C. again. 3a (513 mg, 5.43 mmol) was added to the reaction solution dropwise and the resulting mixture was stirred for 30 min at −78° C., at which point LCMS showed the reaction was finished. The reaction was quenched with saturated aqueous ammonium chloride (5 mL). Ethyl acetate (10 mL*3) was added to the reaction solution to separate the organic layer. The organic layers were combined, dried (Na₂SO₄), filtered and concentrated to give A6 (1.40 g, crude), which was used in the next step without further purification. ¹H NMR (400 MHz, CDCl₃) δ:7.70 (br. s., 1H), 6.86-7.27 (m, 5H), 6.84 (br. s, 1H), 3.84 (s, 3H), 2.76-2.82 (m, 2H), 2.41 (s, 3H), 1.19 (t, J=7.6 Hz, 3H).

### Step 4: N-(2-ethyl-6-methylphenyl)-4-(pyrrolidine-1-carbonyl)pyridine-3-sulfonamide (Compound 101)

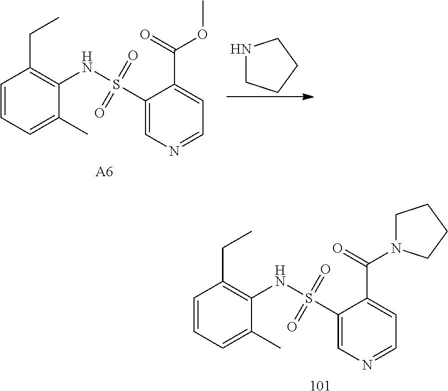

A solution of A6 (900 mg, 2.69 mmol) in pyrrolidine (4.00 mL) was stirred at 50° C. for 2 hrs, at which point LCMS indicated formation of the desired product. The reaction mixture was concentrated under reduced pressure to remove pyrrolidine, and the residue was purified by prep-HPLC (TFA) to give Compound 101 (16.0 mg, yield: 1.99%) as a white solid. ¹H NMR (400 MHz, CDCl₃) δ: 8.92 (s, 1H), 8.88 (d, J=13.2 Hz, 1H), 7.46 (s, 1H), 7.37 (d, J=4.0 Hz, 1H), 7.01-7.16 (m, 3H), 3.73 (t, J=6.8 Hz, 2H), 3.73 (t, J=6.8 Hz, 2H), 2.52 (s, 2H), 2.06 (s, 3H), 1.95-2.01 (m, 4H), 1.06 (t, J=7.2 Hz, 3H).

### Example 3. Dose Response Assay for TDP-43 Inhibition

Exemplary compounds of the invention were evaluated for efficacy in inhibiting TDP-43 inclusions using a dose response assay. Briefly, PC12 cells stably expressing wild type (WT) TDP-43-GFP were stressed with 15 μM to induce TDP-43 inclusions. The cells were then treated with exemplary compounds of the invention and the inhibitory effect on TDP-43 inclusions was observed using fluorescent microscopy. The ratio of cells with TDP-43 inclusions was calculated based on the total number of cells with detectable GFP expression. A 12-point dose response curve was generated, and the IC₅₀ for each compound tested was determined. Results of the dose response assay for exemplary compounds of the invention are summarized in Table 1, wherein A represents an IC₅₀ value of <100 nM; B represents an IC₅₀ value of 101-250 nM; C represents an IC₅₀ value of 251-500 nM; D represents an IC₅₀ value of >500 nM; and ND signifies that the IC₅₀ value was not determined.

### Example 4. Neuroprotection Assay

### Assay Media:

CMF Dissection Buffer:

1× Hank's balanced salt solution (Ca—/Mg, 500 mL) and 10 mM HEPES, pH 7.25-7.3 (IM stock, 5 mL)

Plating Media:

MEM (Earle salts+/Glutamine, 95 mL), FBS (to 2.5%, 2.5 mL), Pen/Strep (1×, 1 mL), glutamine (1×, 1 mL), and D-glucose (0.6% w/v, 0.6 g)

Feeding Media:

neurobasal media (96 mL), B27 supplement (2 mL), Pen/Strep (1 mL), and glutamine (1 mL).

### Procedure:

Embryonic mouse hippocampal neurons were cultured according to Kaech, S. and Banker, G. (2006) Nat Protoc 1:2406-2415 and dissected at P0 from CD1 mice. Once all the hippocampi were removed, they were placed in a 15 mL conical Falcon tube on ice and brought to a final volume of 4.5 mL with CMF dissection buffer. 0.5 mL of a 2.5% trypsin-EDTA solution was then added, and the mixture was incubated at 37° C. for 15 min. The trypsin solution was gently removed, leaving the tissue at the bottom of the Falcon tube. 5 mL CMF dissection buffer was then added, and after gentle mixing, the tissue was allowed to sediment. This procedure was repeated three times. The hippocampi were then dissociated by adding 1.8 mL platting media and repeatedly pipetting in a glass Pasteur pipette; the dissociation process was repeated 5-10 times. The cells were then passed through a 70 um cell strainer into a 50 mL conical tube to remove clumps and debris, and the neurons were plated on glass coverslips coated with poly-D-lysine/laminin. On DIV 1 neurons were transduced with AAV1 EGFP, WT TDP-43 EGFP, A315T TDP-43 EGFP, or Q331K TDP-43 EGFP. Starting at DIV7 neurons were treated every 48 h (DIV7, 9, 11) with an exemplary compound of the invention at a concentration of 10 times the IC₅₀ value. On DIV12, neurons were fixed in 4% PFA and stained for MAP2 or β-3-tubulin (0.1% Triton-X100 antigen retrieval, block in 10% Donkey Serum, primary overnight 1:1000 (Aves) or 1:500 (Millipore) at 4° C. in 5% Donkey Serum). Imaging was done on the Zeiss microscope at 20× with 6×6 tiling. Neurons were traced and analyzed using NeuronJ.

Results of the neuroprotection assay for exemplary compounds of the invention are summarized in Table 1, wherein A represents an average rescue total dendrite length of >150%; B represents an average rescue total dendrite length of 100-149%; C represents an average rescue total dendrite length of 50-99%; D represents an average rescue total dendrite length of 0-49%; E represents an average rescue total dendrite length of <0%; and ND signifies that the average rescue total dendrite length was not determined.

TABLE 1
Efficacy of Exemplary Compounds of the Invention

| | | Average Additive |
|---|---|---|
| Compound No. | IC₅₀ (nM) | Dendrite Length (%) |
| 100 | D | C |
| 101 | A | C |
| 102 | B | ND |

### EQUIVALENTS

It will be recognized that one or more features of any embodiments disclosed herein may be combined and/or rearranged within the scope of the invention to produce further embodiments that are also within the scope of the invention.

Those skilled in the art will recognize, or be able to ascertain using no more than routine experimentation, many equivalents to the specific embodiments of the invention described herein. Such equivalents are intended to be within the scope of the present invention.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and/or rearranged in various ways within the scope and spirit of the invention to produce further embodiments that are also within the scope of the invention. Those skilled in the art will recognize, or be able to ascertain, using no more than routine experimentation, numerous equivalents to the specific embodiments described specifically in this disclosure. Such equivalents are intended to be encompassed in the scope of the following claims.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

1. A compound of Formula (I): 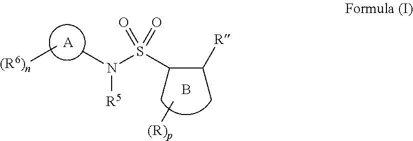
or a pharmaceutically acceptable salt thereof, wherein:
Ring A is aryl or heteroaryl;
Ring B is 6-membered aryl or 5- or 6-membered heteroaryl;
R″ is H or C(O)R¹;
R¹ is C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cycloalkyl, heterocyclyl, aryl, heteroaryl, arylalkyl, heteroarylalkyl, cycloalkylalkyl, heterocyclylalkyl, —OR^{A}, —NR^{B}R^{C}, —NR^{B}C(O)R^{D}, or —SR^{E}, each of which is optionally substituted with 1-5 R⁷;
each R is independently H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cyano, nitro, cycloalkyl, heterocyclyl, aryl, heteroaryl, arylalkyl, heteroarylalkyl, cycloalkylalkyl, heterocyclylalkyl, —OR^{A}, —NR^{B}R^{C}, —C(O)R^{D}, —C(O)OR^{A}, —C(O)NR^{B}R^{C}, —NR^{B}C(O)R^{D}, —NR^{B}C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁸;
R⁵ is H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, —C(O)R^{D}, —C(O)OR^{A}, —C(O)NR^{B}R^{C}, —OR^{A}, or —SR^{E}, each of which is optionally substituted with 1-5 R⁹;
or R⁵, together with the nitrogen atom to which it is attached, forms a heterocyclyl or heteroaryl ring with Ring A, optionally substituted with 1-3 R⁹;
each R⁶ is independently C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, cycloalkylalkyl, heterocyclylalkyl, cyano, hydroxy, halo, —OR^{A}, —NR^{B}R^{C}, —C(O)R^{D}, —C(O)OR^{A}, —C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁹;
each R^{A}, R^{B}, R^{C}, R^{D}, or R^{E} is independently H, C₁-C₆ alkyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, cycloalkylalkyl, heterocycloalkyl, each of which is optionally substituted with 1-4 occurrences of R⁷; or R^{B} and R^{C}, together with the atoms to which each is attached, form a heterocyclyl ring optionally substituted with 1-4 R⁷;
each R⁷, R⁸, or R⁹ is independently C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, halo, cyano, nitro, —OR^{a}, —NR^{b}R^{c}, —C(O)R^{d}, —C(O)OR^{a}, —C(O)NR^{b}R^{c}, —NR^{b}C(O)R^{d}, —NR^{b}C(O)NR^{b}R^{c}, —SR^{e}, —S(O)R^{e}, —S(O)₂R^{e}, —NR^{b}S(O)₂R^{e}, or —S(O)₂NR^{b}R^{c}, each of which is optionally substituted with 1-5 R¹⁰;
each R¹⁰ is C₁-C₆ alkyl, C₁-C₆ heteroalkyl, C₁-C₆ haloalkyl, C₁-C₆ hydroxyalkyl, C₁-C₆ alkoxy, C₁-C₆ haloalkoxy, cycloalkyl, heterocyclyl, aryl, heteroaryl, halo, hydroxy, cyano, or nitro, each of which is optionally substituted with 1-4 R¹¹;
each R^{a}, R^{b}, R^{c}, R^{d}, or R^{e} is H, C₁-C₆ alkyl, cycloalkyl, heterocyclyl, aryl, or heteroaryl, each of which is optionally substituted with R¹¹; or R^{B} and R^{C}, together with the atoms to which each is attached, form a cycloalkyl or heterocyclyl ring optionally substituted with 1-4 R¹¹;
each R¹¹ is independently C₁-C₆ alkyl, halo, hydroxy, cycloalkyl, alkoxy, keto, cyano, or nitro;
n is 0, 1, 2, 3, 4, or 5; and
p is 0, 1, or 2;
provided that Ring B is not 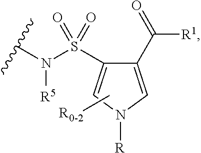
wherein the connection to C(O)R¹ and N(R⁵)S(O)₂— is as shown.

2. The compound of claim 1, wherein R″ is H or C(O)R¹.

3. (canceled)

4. The compound of claim 1, wherein R¹ is C₁-C₆ alkyl, C₂-C₆ alkenyl, cycloalkyl, —OR^{A}, or —NR^{B}R^{C}, and R^{B} and R^{C}, together with the atoms to which each is attached, form a heterocyclyl ring optionally substituted with 1-4R⁷.

5. The compound of claim 1, wherein R¹ is —NR^{B}R^{C}, and R^{B} and R^{C}, together with the atoms to which each is attached, form a heterocyclyl ring optionally substituted with 1-4 R⁷.

6. The compound of claim 1, wherein R¹ is selected from the group consisting of

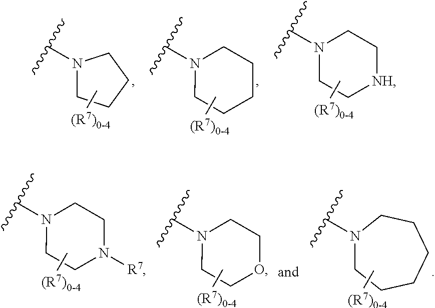

7. (canceled)

8. The compound of claim 1, wherein each R is independently H, C₁-C₆ alkyl, C₂-C₆ alkenyl, C₁-C₆ heteroalkyl, C₁-C₆ haloalkyl, C₁-C₆ hydroxyalkyl, halo, cycloalkyl, heterocyclyl, aryl, heteroaryl, —OR^{A}, —NR^{B}R^{C}, —C(O)R^{D}, —C(O)OR^{A}, —C(O)NR^{B}R^{C}, or —NR^{B}C(O)R^{D}, each of which is optionally substituted with 1-5 R⁸.

9. The compound of claim 1, wherein each R is independently H, C₁-C₆ alkyl, C₁-C₆ haloalkyl, or halo, each of which is optionally substituted with 1-5 R⁸.

10. The compound of claim 1, wherein R is H, C₁-C₆ alkyl, C₂-C₆ alkenyl, C₁-C₆ heteroalkyl, C₁-C₆ hydroxyalkyl, cycloalkyl, heterocyclyl, aryl, or heteroaryl, each of which is optionally substituted with 1-5 R⁸.

11. (canceled)

12. The compound of claim 1, wherein R⁵ is H, C₁-C₆ alkyl, C₂-C₆ alkenyl, C₁-C₆ heteroalkyl, C₁-C₆ haloalkyl, C₁-C₆ hydroxyalkyl, cycloalkyl, heterocyclyl, aryl, or heteroaryl, each of which is optionally substituted with 1-5 R⁹.

13. (canceled)

14. The compound of claim 1, wherein Ring A is a monocyclic or bicyclic aryl or heteroaryl.

15. (canceled)

16. The compound of claim 1, wherein Ring A is phenyl or a 5- or 6-membered heteroaryl.

17-18. (canceled)

19. The compound of claim 1, wherein R⁶ is C₁-C₆ alkyl, C₂-C₆ alkenyl, C₁-C₆ heteroalkyl, C₁-C₆ haloalkyl, C₁-C₆ hydroxyalkyl, cycloalkyl, heterocyclyl, aryl, heteroaryl, cyano, hydroxy, halo, —OR^{A}, —NR^{B}R^{C}, —C(O)R^{D}, —C(O)OR^{A}, —C(O)NR^{B}R^{C}, —S(O)₂R^{E}, —NR^{B}S(O)₂R^{E}, or —S(O)₂NR^{B}R^{C}, each of which is optionally substituted with 1-5 R⁹.

20-21. (canceled)

22. The compound of claim 1, wherein Ring A is selected from the group consisting of:

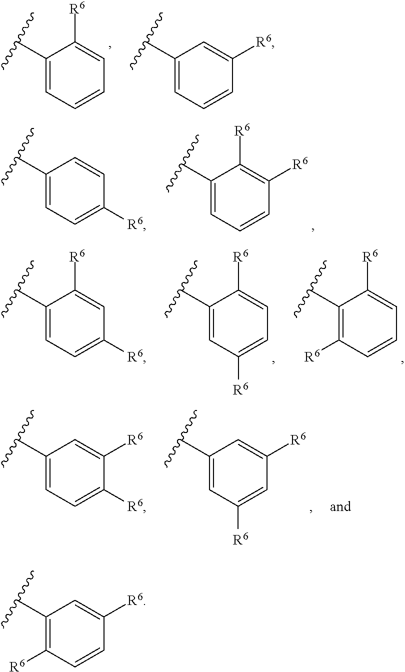

23. (canceled)

24. The compound of claim 22, wherein Ring A is 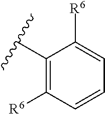
and R⁶ is C₁-C₆ alkyl or halo.

25. (canceled)

26. The compound of claim 1, wherein Ring B is phenyl.

27. The compound of claim 1, wherein Ring B is selected from

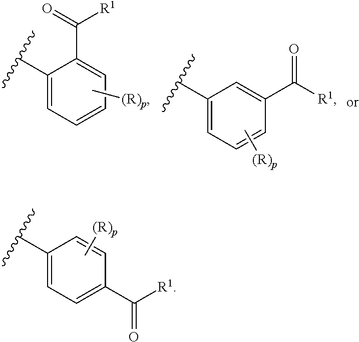

28. The compound of claim 1, wherein Ring B is a 5- or 6-membered heteroaryl.

29. (canceled)

30. The compound of claim 28, wherein Ring B is selected from 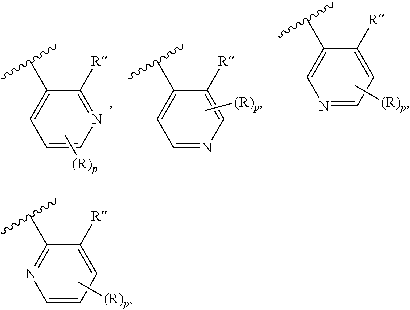
wherein the connectivity to —S(O)₂ is indicated by a wavy line and the connectivity to R″ is as shown.

31. The compound of claim 1, wherein n is 0, 1, or 2 and p is 0 or 1.

32. (canceled)

33. The compound of claim 1, wherein the compound of Formula (I) is a compound of Formula (Ib): 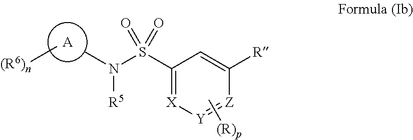
or a pharmaceutically acceptable salt thereof, wherein each of X, Y, and Z is independently N or C(R^{D})₂, and Ring A, R, R″, R⁵, R⁶, R^{D}, n, p and subvariables thereof are defined as for Formula (I).

34-36. (canceled)

37. The compound of claim 1, wherein the compound of Formula (I) is selected from the group consisting of: 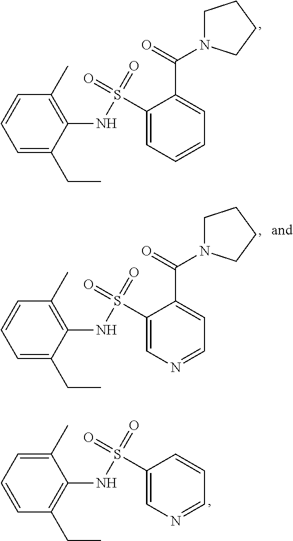
or a pharmaceutically acceptable salt thereof.

38. A pharmaceutical composition comprising a compound of Formula (I) as described in claim 1 or a pharmaceutically acceptable salt thereof in a mixture with a pharmaceutically acceptable excipient, diluent or carrier.

39-43. (canceled)

44. A method for modulating TDP-43 inclusion formation in a subject, the method comprising administering to the subject a compound of Formula (I): 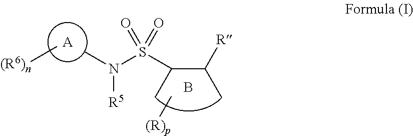
or a pharmaceutically acceptable salt thereof, wherein:
Ring A is aryl or heteroaryl;
Ring B is 6-membered aryl or 5- or 6-membered heteroaryl;
R″ is H or C(O)R¹;
R¹ is C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cycloalkyl, heterocyclyl, aryl, heteroaryl, arylalkyl, heteroarylalkyl, cycloalkylalkyl, heterocyclylalkyl, —OR^{A}, —NR^{B}R^{C}, —NR^{B}C(O)R^{D}, or —SR^{E}, each of which is optionally substituted with 1-5 R⁷;
each R is independently H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cyano, nitro, cycloalkyl, heterocyclyl, aryl, heteroaryl, arylalkyl, heteroarylalkyl, cycloalkylalkyl, heterocyclylalkyl, —OR^{A}, —NR^{B}R^{C}, —C(O)R^{D}, —C(O)OR^{A}, —C(O)NR^{B}R^{C}, —NR^{B}C(O)R^{D}, —NR^{B}C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁸;
R⁵ is H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, —C(O)R^{D}, —C(O)OR^{A}, —C(O)NR^{B}R^{C}, —OR^{A}, or —SR^{E}, each of which is optionally substituted with 1-5 R⁹;
or R⁵, together with the nitrogen atom to which it is attached, forms a heterocyclyl or heteroaryl ring with Ring A, optionally substituted with 1-3 R⁹;
each R⁶ is independently C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, cycloalkylalkyl, heterocyclylalkyl, cyano, hydroxy, halo, —OR^{A}, —NR^{B}R^{C}, —C(O)R^{D}, —C(O)OR^{A}, —C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁹;
each R^{A}, R^{B}, R^{C}, R^{D}, or R^{E} is independently H, C₁-C₆ alkyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, cycloalkylalkyl, heterocycloalkyl, each of which is optionally substituted with 1-4 occurrences of R⁷; or R^{B} and R^{C}, together with the atoms to which each is attached, form a heterocyclyl ring optionally substituted with 1-4 R⁷;
each R⁷, R⁸, or R⁹ is independently C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, halo, cyano, nitro, —OR^{a}, —NR^{b}R^{c}, —C(O)R^{d}, —C(O)OR^{a}, —C(O)NR^{b}R^{c}, —NR^{b}C(O)R^{d}, —NR^{b}C(O)NR^{b}R^{c}, —SR^{e}, —S(O)R^{e}, —S(O)₂R^{e}, —NR^{b}S(O)₂R^{c}, or —S(O)₂NR^{b}R^{c}, each of which is optionally substituted with 1-5 R¹⁰;
each R¹⁰ is C₁-C₆ alkyl, C₁-C₆ heteroalkyl, C₁-C₆ haloalkyl, C₁-C₆ hydroxyalkyl, C₁-C₆ alkoxy, C₁-C₆ haloalkoxy, cycloalkyl, heterocyclyl, aryl, heteroaryl, halo, hydroxy, cyano, or nitro, each of which is optionally substituted with 1-4 R¹¹;
each R^{a}, R^{b}, R^{c}, R^{d}, or R^{e} is H, C₁-C₆ alkyl, cycloalkyl, heterocyclyl, aryl, or heteroaryl, each of which is optionally substituted with R¹¹; or R^{B} and R^{C}, together with the atoms to which each is attached, form a cycloalkyl or heterocyclyl ring optionally substituted with 1-4 R¹¹;
each R¹¹ is independently C₁-C₆ alkyl, halo, hydroxy, cycloalkyl, alkoxy, keto, cyano, or nitro;
n is 0, 1, 2, 3, 4, or 5; and
p is 0, 1, or 2;
provided that Ring B is not 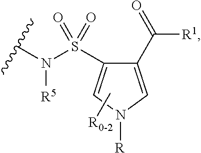
wherein the connection to C(O)R¹ and N(R⁵)S(O)₂— is as shown.

45. The method of claim 44, wherein TDP-43 inclusion formation is inhibited or stimulated.

46. The method of claim 44, wherein TDP-43 inclusion is disaggregated.

47. (canceled)

48. The method of claim 44, wherein the subject is suffering from a neurodegenerative disease or disorder, a musculoskeletal disease or disorder, a cancer, an ophthalmological disease or disorder, and/or a viral infection.

49. The method of claim 48, wherein the neurodegenerative disease is selected from the group consisting of Alzheimer's disease, frontotemporal dementia (FTD), FTLD-U, FTD caused by mutations in the progranulin protein or tau protein (e.g., progranulin-deficient FTLD), frontotemporal dementia with inclusion body myopathy (IBMPFD), frontotemporal dementia with motor neuron disease, amyotrophic lateral sclerosis (ALS), Huntington's disease (HD), Huntington's chorea, prion diseases (e.g., Creutzfeld-Jacob disease, bovine spongiform encephalopathy, Kuru, or scrapie), Lewy Body disease, diffuse Lewy body disease (DLBD), polyglutamine (polyQ)-repeat diseases, trinucleotide repeat diseases, cerebral degenerative diseases, presenile dementia, senile dementia, Parkinsonism linked to chromosome 17 (FTDP-17), progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, HIV-associated dementia, Parkinson's disease, Parkinson's disease with dementia, dementia with Lewy bodies, Down's syndrome, multiple system atrophy, spinal muscular atrophy (SMA, e.g., SMA Type I (e.g., Werdnig-Hoffmann disease) SMA Type II, SMA Type III (e.g., Kugelberg-Welander disease), or congenital SMA with arthrogryposis), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, age-related disorders and dementias, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), autism, or any combination thereof.

50. The method of claim 48, wherein the musculoskeletal disease is selected from the group consisting of muscular dystrophy, facioscapulohumeral muscular dystrophy (e.g., FSHD1 or FSHD2), Freidrich's ataxia, progressive muscular atrophy (PMA), mitochondrial encephalomyopathy (MELAS), multiple sclerosis, inclusion body myopathy, inclusion body myositis (e.g., sporadic inclusion body myositis), post-polio muscular atrophy (PPMA), motor neuron disease, myotonia, myotonic dystrophy, sacropenia, multifocal motor neuropathy, inflammatory myopathies, and paralysis.

51. The method of claim 48, wherein the cancer is selected from the group consisting of breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, ophthalmological cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, vulval cancer, or any combination thereof.

52-53. (canceled)

54. The method of claim 48, wherein the ophthalmological disease is selected from the group consisting of macular degeneration, age-related macular degeneration, diabetes retinopathy, histoplasmosis, macular hole, macular pucker, Bietti's crystalline dystrophy, retinal detachment, retinal thinning, retinoblastoma, retinopathy of prematurity, Usher's syndrome, vitreous detachment, Refsum disease, retinitis pigmentosa, onchocerciasis, choroideremia, Leber congenital amaurosis, retinoschisis, juvenile retinoschisis, Stargardt disease, ophthalmoplegia, or any combination thereof.

55. The method of claim 48, wherein the viral infection is caused by a virus selected from the group consisting of West Nile virus, respiratory syncytial virus (RSV), herpes simplex virus 1, herpes simplex virus 2, Epstein-Barr virus (EBV), hepatitis virus A, hepatitis virus B, hepatitis virus C, influenza viruses, chicken pox, avian flu viruses, smallpox, polio viruses, HIV-1, HIV-2, Ebola virus, and any combination thereof.

56. The method of claim 48, wherein the subject is a mammal.

57-60. (canceled)

61. A method for treating a B-cell or T-cell lymphoma in a subject in need thereof with a compound of Formula (I): 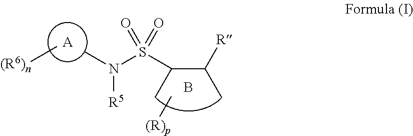
or a pharmaceutically acceptable salt thereof, wherein:
Ring A is aryl or heteroaryl;
Ring B is 6-membered aryl or 5- or 6-membered heteroaryl;
R″ is H or C(O)R¹;
R¹ is C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cycloalkyl, heterocyclyl, aryl, heteroaryl, arylalkyl, heteroarylalkyl, cycloalkylalkyl, heterocyclylalkyl, —OR^{A}, —NR^{B}R^{C}, —NR^{B}C(O)R^{D}, or —SR^{E}, each of which is optionally substituted with 1-5 R⁷;
each R is independently H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cyano, nitro, cycloalkyl, heterocyclyl, aryl, heteroaryl, arylalkyl, heteroarylalkyl, cycloalkylalkyl, heterocyclylalkyl, —OR^{A}, —NR^{B}R^{C}, —C(O)R^{D}, —C(O)OR^{A}, —C(O)NR^{B}R^{C}, —NR^{B}C(O)R^{D}, —NR^{B}C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁸;
R⁵ is H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, —C(O)R^{D}, —C(O)OR^{A}, —C(O)NR^{B}R^{C}, —OR^{A}, or —SR^{E}, each of which is optionally substituted with 1-5 R⁹;
or R⁵, together with the nitrogen atom to which it is attached, forms a heterocyclyl or heteroaryl ring with Ring A, optionally substituted with 1-3 R⁹;
each R⁶ is independently C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, cycloalkylalkyl, heterocyclylalkyl, cyano, hydroxy, halo, —OR^{A}, —NR^{B}R^{C}, —C(O)R^{D}, —C(O)OR^{A}, —C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁹;
each R^{A}, R^{B}, R^{C}, R^{D}, or R^{E} is independently H, C₁-C₆ alkyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, cycloalkylalkyl, heterocycloalkyl, each of which is optionally substituted with 1-4 occurrences of R⁷; or R^{B} and R^{C}, together with the atoms to which each is attached, form a heterocyclyl ring optionally substituted with 1-4 R⁷;
each R⁷, R⁸, or R⁹ is independently C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, halo, cyano, nitro, —OR^{a}, —NR^{b}R^{c}, —C(O)R^{d}, —C(O)OR^{a}, —C(O)NR^{b}R^{c}, —NR^{b}C(O)R^{d}, —NR^{b}C(O)NR^{b}R^{c}, —SR^{e}, —S(O)R^{e}, —S(O)₂R^{e}, —NR^{b}S(O)₂R^{e}, or —S(O)₂NR^{b}R^{c}, each of which is optionally substituted with 1-5 R¹⁰;
each R¹⁰ is C₁-C₆ alkyl, C₁-C₆ heteroalkyl, C₁-C₆ haloalkyl, C₁-C₆ hydroxyalkyl, C₁-C₆ alkoxy, C₁-C₆ haloalkoxy, cycloalkyl, heterocyclyl, aryl, heteroaryl, halo, hydroxy, cyano, or nitro, each of which is optionally substituted with 1-4 R¹¹;
each R^{a}, R^{b}, R^{c}, R^{d}, or R^{e} is H, C₁-C₆ alkyl, cycloalkyl, heterocyclyl, aryl, or heteroaryl, each of which is optionally substituted with R¹¹; or R^{B} and R^{C}, together with the atoms to which each is attached, form a cycloalkyl or heterocyclyl ring optionally substituted with 1-4 R¹¹;
each R¹¹ is independently C₁-C₆ alkyl, halo, hydroxy, cycloalkyl, alkoxy, keto, cyano, or nitro;
n is 0, 1, 2, 3, 4, or 5; and
p is 0, 1, or 2.

62. The method claim 61, wherein the B-cell or T-cell lymphoma is selected from the group consisting of diffuse large B-cell lymphoma, primary mediastinal B-cell lymphoma, intravascular large B-cell lymphoma, follicular lymphoma, chronic lymphocytic leukemia/small lymphocytic lymphoma, mantle cell lymphoma, marginal zone B-cell lymphomas, extranodal marginal B-cell lymphomas, mucosa-associated lymphoid tissue (MALT) lymphomas, modal marginal zone B-cell lymphoma, splenic marginal zone B-cell lymphoma, Burkitt lymphoma, lymphoplasmacytic lymphoma, Waldenström's macroglobulinemia, hairy cell leukemia, primary central nervous system (CNS) lymphoma, precursor T-lymphoblastic lymphoma/leukemia, peripheral T-cell lymphoma, smoldering adult T-cell lymphoma, chronic adult T-cell lymphoma, acute adult T-cell lymphoma, lymphomatous adult T-cell lymphoma, angioimmunoblastic T-cell lymphoma, extranodal natural killer T-cell lymphoma nasal type (ENKL), enteropathy-associated intestinal T-cell lymphoma (EATL), and anaplastic large cell lymphoma (ALCL).

63. The method of claim 61, wherein the compound of Formula (I) is a compound of Formula (Ib): 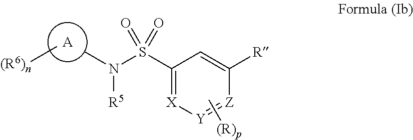
or a pharmaceutically acceptable salt thereof, wherein each of X, Y, and Z is independently N or C(R^{D})₂, and Ring A, R, R″, R⁵, R⁶, n, p and subvariables thereof are defined as for Formula (I).

64. The method of claim 61, wherein the compound of Formula (I) is 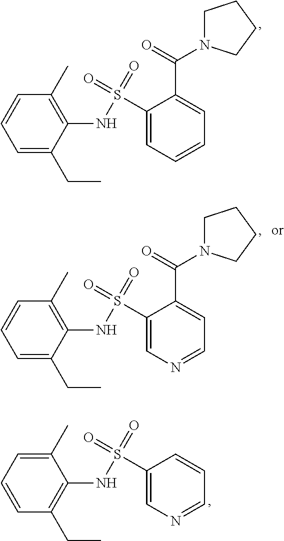
or a pharmaceutically acceptable salt thereof. or a pharmaceutically acceptable salt thereof.

65. A method for treating a neurodegenerative disease selected from the group consisting of frontotemporal dementia caused by mutations in the progranulin protein or tau protein (e.g., progranulin-deficient FTLD), frontotemporal dementia with inclusion body myopathy (IBMPFD), frontotemporal dementia with motor neuron disease, bovine spongiform encephalopathy, Kuru, scrapie, Lewy Body disease, diffuse Lewy body disease (DLBD), polyglutamine (polyQ)-repeat diseases, progressive bulbar palsy (PBP), psuedobulbar palsy, spinal and bulbar muscular atrophy (SBMA), primary lateral sclerosis, HIV-associated dementia, progressive spinobulbar muscular atrophy (e.g., Kennedy disease), post-polio syndrome (PPS), pantothenate kinase-associated neurodegeneration (PANK), Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury) or autism in a subject in need thereof with a compound of Formula (I): 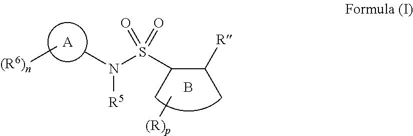
or a pharmaceutically acceptable salt thereof, wherein:
Ring A is aryl or heteroaryl;
Ring B is 6-membered aryl or 5- or 6-membered heteroaryl;
R″ is H or C(O)R¹;
R¹ is C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cycloalkyl, heterocyclyl, aryl, heteroaryl, arylalkyl, heteroarylalkyl, cycloalkylalkyl, heterocyclylalkyl, —OR^{A}, —NR^{B}R^{C}, —NR^{B}C(O)R^{D}, or —SR^{E}, each of which is optionally substituted with 1-5 R⁷;
each R is independently H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cyano, nitro, cycloalkyl, heterocyclyl, aryl, heteroaryl, arylalkyl, heteroarylalkyl, cycloalkylalkyl, heterocyclylalkyl, —OR^{A}, —NR^{B}R^{C}, —C(O)R^{D}, —C(O)OR^{A}, —C(O)NR^{B}R^{C}, —NR^{B}C(O)R^{D}, —NR^{B}C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁸;
R⁵ is H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, —C(O)R^{D}, —C(O)OR^{A}, —C(O)NR^{B}R^{C}, —OR^{A}, or —SR^{E}, each of which is optionally substituted with 1-5 R⁹;
or R⁵, together with the nitrogen atom to which it is attached, forms a heterocyclyl or heteroaryl ring with Ring A, optionally substituted with 1-3 R⁹;
each R⁶ is independently C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, cycloalkylalkyl, heterocyclylalkyl, cyano, hydroxy, halo, —OR^{A}, —NR^{B}R^{C}, —C(O)R^{D}, —C(O)OR^{A}, —C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁹;
each R^{A}, R^{B}, R^{C}, R^{D}, or R^{E} is independently H, C₁-C₆ alkyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, cycloalkylalkyl, heterocycloalkyl, each of which is optionally substituted with 1-4 occurrences of R⁷; or R^{B} and R^{C}, together with the atoms to which each is attached, form a heterocyclyl ring optionally substituted with 1-4 R⁷;
each R⁷, R⁸, or R⁹ is independently C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, halo, cyano, nitro, —OR^{a}, —NR^{b}R^{c}, —C(O)R^{d}, —C(O)OR^{a}, —C(O)NR^{b}R^{c}, —NR^{b}C(O)R^{d}, —NR^{b}C(O)NR^{b}R^{c}, —SR^{e}, —S(O)R^{e}, —S(O)₂R^{e}, —NR^{b}S(O)₂R^{e}, or —S(O)₂NR^{b}R^{c}, each of which is optionally substituted with 1-5 R¹⁰;
each R¹⁰ is C₁-C₆ alkyl, C₁-C₆ heteroalkyl, C₁-C₆ haloalkyl, C₁-C₆ hydroxyalkyl, C₁-C₆ alkoxy, C₁-C₆ haloalkoxy, cycloalkyl, heterocyclyl, aryl, heteroaryl, halo, hydroxy, cyano, or nitro, each of which is optionally substituted with 1-4 R¹¹;
each R^{a}, R^{b}, R^{c}, R^{d}, or R^{e} is H, C₁-C₆ alkyl, cycloalkyl, heterocyclyl, aryl, or heteroaryl, each of which is optionally substituted with R¹¹; or R^{B} and R^{C}, together with the atoms to which each is attached, form a cycloalkyl or heterocyclyl ring optionally substituted with 1-4 R¹¹;
each R¹¹ is independently C₁-C₆ alkyl, halo, hydroxy, cycloalkyl, alkoxy, keto, cyano, or nitro;
n is 0, 1, 2, 3, 4, or 5; and
p is 0, 1, or 2;
provided that Ring B is not 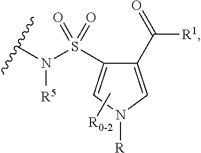
wherein the connection to C(O)R¹ and N(R⁵)S(O)₂— is as shown.

66. The method of claim 65, wherein the compound of Formula (I) is a compound of Formula (Ib): 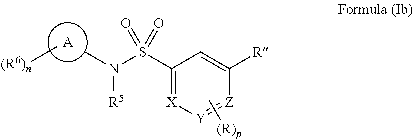
or a pharmaceutically acceptable salt thereof, wherein each of X, Y, and Z is independently N or C(R^{D})₂, and Ring A, R, R″, R⁵, R⁶, n, p and subvariables thereof are defined as for Formula (I).

67. The method of claim 65, wherein the compound of Formula (I) is 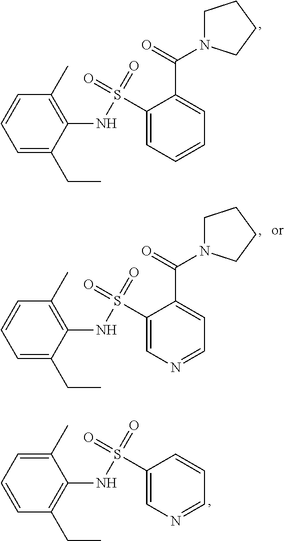
or a pharmaceutically acceptable salt thereof. or a pharmaceutically acceptable salt thereof.

68. A method for treating a musculoskeletal disease in a subject in need thereof with a compound of Formula (I): 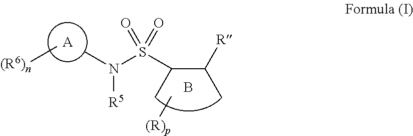
or a pharmaceutically acceptable salt thereof, wherein:
Ring A is aryl or heteroaryl;
Ring B is 6-membered aryl or 5- or 6-membered heteroaryl;
R″ is H or C(O)R¹;
R¹ is C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cycloalkyl, heterocyclyl, aryl, heteroaryl, arylalkyl, heteroarylalkyl, cycloalkylalkyl, heterocyclylalkyl, —OR^{A}, —NR^{B}R^{C}, —NR^{B}C(O)R^{D}, or —SR^{E}, each of which is optionally substituted with 1-5 R⁷;
each R is independently H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, halo, cyano, nitro, cycloalkyl, heterocyclyl, aryl, heteroaryl, arylalkyl, heteroarylalkyl, cycloalkylalkyl, heterocyclylalkyl, —OR^{A}, —NR^{B}R^{C}, —C(O)R^{D}, —C(O)OR^{A}, —C(O)NR^{B}R^{C}, —NR^{B}C(O)R^{D}, —NR^{B}C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁸;
R⁵ is H, C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, —C(O)R^{D}, —C(O)OR^{A}, —C(O)NR^{B}R^{C}, —OR^{A}, or —SR^{E}, each of which is optionally substituted with 1-5 R⁹;
or R⁵, together with the nitrogen atom to which it is attached, forms a heterocyclyl or heteroaryl ring with Ring A, optionally substituted with 1-3 R⁹;
each R⁶ is independently C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, cycloalkylalkyl, heterocyclylalkyl, cyano, hydroxy, halo, —OR^{A}, —NR^{B}R^{C}, —C(O)R^{D}, —C(O)OR^{A}, —C(O)NR^{B}R^{C}, —SR^{E}, —S(O)R^{E}, —S(O)₂R^{E}, —NR^{B}S(O)₂R^{E}, or —S(O)₂NR^{B}R^{C}, each of which is optionally substituted with 1-5 R⁹;
each R^{A}, R^{B}, R^{C}, R^{D}, or R^{E} is independently H, C₁-C₆ alkyl, C₁-C₆ heteroalkyl, C₁-C₆ haloalkyl, C₁-C₆ hydroxyalkyl, cycloalkyl, heterocyclyl, aryl, heteroaryl, arylalkyl, heteroarylalkyl, cycloalkylalkyl, heterocycloalkyl, each of which is optionally substituted with 1-4 occurrences of R⁷; or R^{B} and R^{C}, together with the atoms to which each is attached, form a heterocyclyl ring optionally substituted with 1-4 R⁷;
each R⁷, R⁸, or R⁹ is independently C₁-C₆ alkyl, C₂-C₆ alkenyl, C₂-C₆ alkynyl, C₁-C₆ heteroalkyl, C₁-C₆ haloalkyl, C₁-C₆ hydroxyalkyl, cycloalkyl, heterocyclyl, aryl, heteroaryl, halo, cyano, nitro, —OR^{a}, —NR^{b}R^{c}, —C(O)R^{d}, —C(O)OR^{a}, —C(O)NR^{b}R^{c}, —NR^{b}C(O)R^{d}, —NR^{b}C(O)NR^{b}R^{c}, —SR^{e}, —S(O)R^{e}, —S(O)₂R^{e}, —NR^{b}S(O)₂R^{e}, or —S(O)₂NR^{b}R^{c}, each of which is optionally substituted with 1-5 R¹⁰;
each R¹⁰ is C₁-C₆ alkyl, C₁-C₆ heteroalkyl, C₁-C₆ haloalkyl, C₁-C₆ hydroxyalkyl, C₁-C₆ alkoxy, C₁-C₆ haloalkoxy, cycloalkyl, heterocyclyl, aryl, heteroaryl, halo, hydroxy, cyano, or nitro, each of which is optionally substituted with 1-4 R¹¹;
each R^{a}, R^{b}, R^{c}, R^{d}, or R^{e} is H, C₁-C₆ alkyl, cycloalkyl, heterocyclyl, aryl, or heteroaryl, each of which is optionally substituted with R¹¹; or R^{B} and R^{C}, together with the atoms to which each is attached, form a cycloalkyl or heterocyclyl ring optionally substituted with 1-4 R¹¹;
each R¹¹ is independently C₁-C₆ alkyl, halo, hydroxy, cycloalkyl, alkoxy, keto, cyano, or nitro;
n is 0, 1, 2, 3, 4, or 5; and
p is 0, 1, or 2.

69. The method of claim 68, wherein Ring B is not

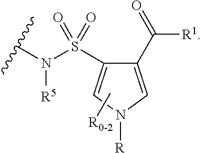

70. The method of claim 68, wherein the musculoskeletal disease is selected from the group consisting of muscular dystrophy, facioscapulohumeral muscular dystrophy (e.g., FSHD1 or FSHD2), Freidrich's ataxia, progressive muscular atrophy (PMA), mitochondrial encephalomyopathy (MELAS), multiple sclerosis, inclusion body myopathy, inclusion body myositis (e.g., sporadic inclusion body myositis), post-polio muscular atrophy (PPMA), motor neuron disease, myotonia, myotonic dystrophy, sacropenia, multifocal motor neuropathy, inflammatory myopathies, and paralysis.

71. The method of claim 68, wherein the compound of Formula (I) is a compound of Formula (Ib): 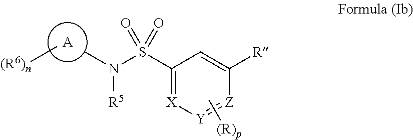
or a pharmaceutically acceptable salt thereof, wherein each of X, Y, and Z is independently N or C(R^{D})₂, and Ring A, R, R″, R⁵, R⁶, n, p and subvariables thereof are defined as for Formula (I).

72. The method of claim 68, wherein the compound of Formula (I) is 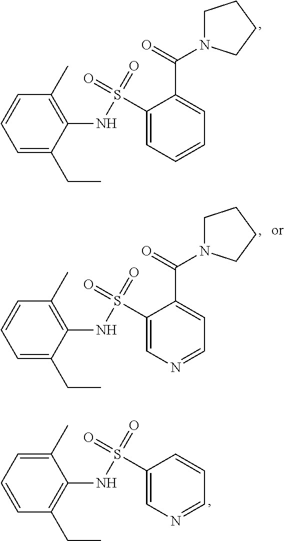
or a pharmaceutically acceptable salt thereof. or a pharmaceutically acceptable salt thereof.
